(19) United States
(12) Patent Application Publication
(10) Pub. No.: US 2021/0372995 A1
(43) Pub. Date: Dec. 2, 2021

(54) BIOMIMETIC PLATFORMS TO MODEL VASCULAR PATHOPHYSIOLOGY, DIAGNOSTICS, AND THERAPY

(71) Applicant: The Johns Hopkins University, Baltimore, MD (US)

(72) Inventors: Lewis Romer, Baltimore, MD (US); David Gracias, Baltimore, MD (US); Jayson Pagaduan, Windsor, MD (US); Anil Bhatta, Baltimore, MD (US); Xing Chen, Baltimore, MD (US); Qianru Jin, Cambridge, MA (US)

(21) Appl. No.: 16/758,030

(22) PCT Filed: Oct. 22, 2018
(86) PCT No.: PCT/US18/56923

§ 371 Date: Apr. 21, 2020

Related U.S. Application Data
(60) Provisional application No. 62/575,492, filed on Oct. 22, 2017.

(51) Int. Cl. G01N 33/50; C12N 5/077; C12N 5/071; B01L 3/00

(52) U.S. Cl. CPC G01N 33/5082; C12N 5/0661; C12N 5/0691; B01L 2200/027; C12N 2533/30; C12N 2535/00; B01L 3/502715

(57) ABSTRACT

In one aspect, provided is a composition (biomimetic composition) that includes a biomimetic in vitro model of an arteriolar vessel comprising: at least one of 1) human smooth muscle cells and 2) human pulmonary endothelial cells; wherein the vessel recapitulates one or more of the overall tubular geometry, morphometrics, extracellular matrix constituents, cellular morphology, cellular alignment, and functional heterotypic connections between the human smooth muscle cells and/or the human endothelial cells as compared to an in vivo arteriolar vessel. A microfluidics-based model platform of the pulmonary circulation is provided. Methods of use include measuring flow in biomimetic vessels, and to determine the resistance of these biomimetic vessels in the setting of a variety of experimental conditions that recapitulate the pathobiology of pulmonary hypertension.

### CROSS REFERENCE TO RELATED APPLICATIONS

This application claims the benefit of U.S. Provisional Application 62/575,492 filed on Oct. 22, 2017, the entire contents of which is incorporated herein by reference in its entirety.

### FIELD OF THE INVENTION

This invention relates generally to the field of therapies and diagnostics for vascular diseases and disorders such as pulmonary hypertension.

### BACKGROUND

Pulmonary arterial hypertension (PAH) is a progressive and often fatal illness of the pulmonary microvasculature. The prevalence of PAH in the US has been estimated at 109 per million among individuals under 65, and 451 per million among those 65 and over, with average survival after diagnosis estimated at 5 to 7 years. Prior to the invention described herein, there were no known cures for this condition, and it has a reported mortality rate of up to 48%. Prior to the invention described herein, fundamental questions regarding mechanisms underlying the pathogenesis of PAH still remained. Animal models of PAH have limitations, as they differ from humans lungs with regard to the anatomy of the pulmonary circulation. As such, prior to the invention described herein, there was an unmet need to develop new strategies for the investigation and treatment of pulmonary arterial hypertension.

### SUMMARY OF THE INVENTION

In one aspect, the invention is based, at least in part, on the surprising discovery that biomimetic platforms and composite microtissues comprised of biomaterials and human cells can be developed for studying functional aspects of microvascular physiology that are relevant to pulmonary hypertension and other vascular disorders and diseases. Methods of use include measuring flow in the biomimetic vessels, and to determine the resistance of these biomimetic vessels in the setting of a variety of experimental conditions that recapitulate the pathobiology of pulmonary hypertension.

In a particular aspect, the invention is based, at least in part, on the surprising discovery that biomimetic platforms and tissues can be developed for studying pulmonary hypertension and other vascular disorders and diseases.

In one aspect, compositions of human smooth muscle cells are provided cultivated on micro-wrinkled sheets to mimic vascular function. In a particular aspect, provided are compositions of human pulmonary artery smooth muscle cells (HPASMC) cultivated on micro-wrinkled elastomeric sheets such as polydimethylsiloxane (PDMS) sheets to mimic pulmonary arteriole structure and function. Furthermore, the invention also provides for a platform that mimics the 3D architecture and function of human pulmonary arterioles. Compositions of the present inventions may be particularly used to examine the pathobiology and potential efficacy of therapies for pulmonary hypertension.

The compositions of the invention are also useful for examining the pathobiology and potential efficacy of therapies for other microvasculature conditions.

In one preferred aspect, provided is a composition (also sometimes referred to herein as a biomimetic composition) that includes a biomimetic in vitro model of an arteriolar vessel comprising: at least one of 1) human smooth muscle cells and 2) human pulmonary endothelial cells; wherein the vessel recapitulates one or more of the overall tubular geometry, morphometrics, extracellular matrix constituents, cellular morphology, cellular alignment, and functional heterotypic connections between the human smooth muscle cells and/or the human endothelial cells as compared to an in vivo arteriolar vessel. Preferably, the model comprises both human smooth muscle cells and human endothelial cells. The biomimetic in vitro model suitably may be e.g. a model of a small blood vessel, ocular vessel, renal vessel or coronary vessel and preferably may be a model of a pulmonary vessel.

In a specifically preferred aspect, provided is a composition (also sometimes referred to herein as a biomimetic composition) that includes a biomimetic in vitro model of a pulmonary vessel comprising: at least one of 1) human pulmonary artery smooth muscle cells (HPASMC) and 2) human pulmonary endothelial cells (HPMEC); wherein the vessel recapitulates one or more of the overall tubular geometry, morphometrics, extracellular matrix constituents, cellular morphology, cellular alignment, and functional heterotypic connections between the HPASMC and the HPMEC as compared to an in vivo pulmonary vessel.

In preferred embodiments, the in vitro models include a monolayer of endothelial cells, for example, a monolayer of HPMEC.

In preferred embodiments, the biomimetic composition further includes elastomeric sheets, wherein the smooth muscle cells such as HPASMC and/or the endothelial cells such as HPMEC are upon the elastomeric sheets. The elastomeric sheets may be micro-wrinkled. The elastomeric sheets may include polydimethylsiloxane (PDMS) or boronic acid-functional poly(amido) amines, although a variety of materials will be suitable. The PDMS sheets may be a variety of dimensions and configurations including e.g. about 5-10 μm thick.

In preferred embodiments, the cellular alignment includes smooth muscle cells such as HPASMC aligned in parallel along a long axis of a micro-wrinkle.

In preferred embodiments, the biomimetic composition further includes a culture media comprising type I collagen or laminin 3 In preferred embodiments, the morphology includes human smooth muscle cells such as HPASMC with an elongated, and stretched morphology. The aligned human smooth muscle cells such as HPASMC may have a smooth muscle contractile phenotype. The smooth muscle contractile phenotype suitably may include an increase in expression of smooth muscle proteins. The smooth muscle proteins suitably may include smooth muscle α-actin.

In preferred embodiments, the functional heterotypic connections mimic myoendothelial junctions (MEJ) in an in vivo pulmonary vessel. The HPMECs may be characterized by hypoxic upregulation of arginase 2. The in vitro model may include reduced NO production with hypoxia. The model may include reduced NO diffusion. The model also may exhibit increased nitric oxide (NO) production by HPMEC in the tubular geometry, increased endothelial nitric oxide synthase (eNOS) phosphorylation, and/or decreased arginase 2 expression.

In preferred embodiments, the functional heterotypic connections are characterized by cell-cell communication which mimics an in vivo pulmonary vessel.

In one aspect, provided herein is a method of screening candidate compounds for treating pulmonary arterial hypertension (PAH) or other microvasculature condition that may include: inducing a biomimetic composition or vessel as described herein to exhibit PAH or other abnormal vasomotor behavior; determining that the vessel or biomimetic composition exhibits PAH or other abnormal vasomotor behavior; administering a candidate compound to the vessel or composition, and determining whether the vessel or biomimetic composition has a reduction in PAH or other abnormal vasomotor behavior relative to a control vessel or biomimetic composition that does not exhibit PAH or other abnormal vasomotor behavior. In preferred embodiments, the induction of PAH includes exposing the vessel or biomimetic composition to hypoxic conditions, endothelin 1, inflammatory cytokines, or other vasoactive peptides. The induction of PAH may include exposing the vessel or biomimetic composition to hypoxic conditions. In preferred embodiments, the PAH behavior may be characterized by diminished NO production. In preferred embodiments, a reduction in PAH may be determined by identifying one or more of an increased production of NO, increased flow, decreased resistance, and/or increased internal diameter.

Further provided herein is a method of producing a biomimetic in vitro model of a pulmonary vessel comprising: cultivating HPASMC on elastomeric sheets; and adding monolayers of endothelial cells, wherein the vessel recapitulates cellular morphology, cellular alignment, and functional heterotypic connections between the HPASMC and the endothelial cells as compared to an in vivo pulmonary vessel. The endothelial cells may include HPMEC.

In other preferred aspect, provided herein is a composition including a biomimetic, three-dimensional (3D), in vitro tubular model of a pulmonary vessel that includes: a biodegradable, self-folding scaffold comprising one or more cardiovascular cells seeded on a substrate, wherein the tubular model closely mimics the diameter of an in vivo pulmonary vessel, the tubular model includes micropatterned fibronectin and HPASMC that are aligned on a patterned substrate, an internal elastic lamina, and a monolayer of HPMEC, and the tubular model promotes cell-cell communication. Suitable substrates include a film of germanium and/or a silicon monoxide/silicon dioxide bilayer, or a layer of another metal, or a layer of an elastomer.

In preferred embodiments, the internal elastic lamina includes laminin 3 In preferred embodiments, the film enables protein immobilization. The protein may include fibronectin. In preferred embodiments, the one or more cardiovascular cells are aortic cells or pulmonary cells, or combinations thereof. The aortic cells may be human telomerase reverse transcriptase-transformed human aortic endothelial cells (hTERT-HAEC). The pulmonary cells may be selected from the group consisting of HPASMC, HPAEC, and co-cultures thereof.

In preferred embodiments, the self-folding scaffolds include rolls of 3D structures with or without chemical etchants. The self-folding scaffolds may include extracellular matrix (ECM) proteins. The ECM proteins may include fibronectin, laminin, collagen, or combinations thereof.

In preferred embodiments, the 3D model includes a lumen diameter of between 10 μm and 1,000 μm. Preferably, the 3D model may include a lumen diameter of between 25 μm and 200 μm. In preferred embodiments, the model includes a mean cell density of between 21-24 cells/4000 m².

In preferred embodiments, the cells remain viable for at least 60 days. The cells may remain viable for 14-30 days.

In preferred embodiments, the cardiovascular cells exhibit smooth muscle cell or endothelial markers. The endothelial markers may be selected from the group consisting of and VE-cadherin; smooth muscle cells exhibit markers including smoothelin, alpha smooth muscle actin, and diphosphorylated myosin light chain.

In preferred embodiments, the model includes ECM which mimics cellular adhesion and cellular proliferation of in vivo pulmonary vessels. In preferred embodiments, the model includes vessels with a Young's modulus of about 0.1 to 15 kPa. In preferred embodiments, the model includes cells with adherent junctions between cells. In preferred embodiments, the model includes endothetial cells (e.g. hTERT-HAEC cells or HPMEC) with increased NO production. In preferred embodiments, the model includes smooth muscle cells with a contractile phenotype.

In preferred embodiments, the model includes structures vessels with microfluidic components. The microfluidic components may comprise tapered channels (device) for atraumatic trapping and dynamic interrogation of biomimetic and harvested ex vivo arterioles. Preferably, the microfluidic components permit optical imaging of the assessed materials. The microfluidic components also may include poly(dimethyl siloxane) (PDMS) or poly(methyl methacrylate), or other suitable materials. Such structures useful in the present biomimetic compositions can be reliably produced using a highly parallel, high throughput processes.

In certain embodiments, the device or model for modeling pulmonary circulation comprises a rectangular platform comprising an input port at an opposing end of an output port for circulation of flow; a microchannel connecting the input and output port wherein the microchannel is tapered having a smaller diameter at the output port; a loading well interposed between the input port and output port, an upstream pressure readout port connected to the microchannel and situated after the loading well; a downstream pressure readout port connected to the microchannel and situated prior to the output port. The input and output ports provide for circulation of flow and the loading well is provided for insertion of 3-dimensional (3D) model vessels. In certain embodiments, the pressure readout ports at the upstream and downstream ends of the microchannel are used to track systolic and diastolic pressures in real-time. The pressure differences between the upstream and downstream pressure readout ports are a measure of vascular resistance across the microchannel. In certain embodiments, the device or model is produced via 3D printing methods. In certain embodiments, the device further comprises a peristatic pump for supplying tunable flow patterns to the microchannel; two or more piezoresistive pressure sensors, connected to the pressure readout ports, for transducing transduce hydrostatic pressure to an electrical resistance signal. The electrical resistance signal is converted to digital data for signal processing.

Other aspects of the invention are disclosed infra.

### Definitions

Unless specifically stated or obvious from context, as used herein, the term “about” is understood as within a range of normal tolerance in the art, for example, within two standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

By “agent” is meant any small compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

By “analog” is meant a molecule that is not identical, but has analogous functional or structural features to those of a corresponding reference molecule. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “binding to” a molecule is meant having a physicochemical affinity for other molecule, which can promote actual physical or chemical interaction between the molecule and the other molecule.

By “microchannel,” as used herein, means a feature on or in a substrate that at least partially directs the flow of a fluid. In some cases, the channel may be formed, at least in part, by a single component, e.g., an etched substrate or molded unit. The channel can have any cross-sectional shape, for example, circular, oval, triangular, irregular, square or rectangular (having any aspect ratio), or the like, and can be covered or uncovered (i.e., open to the external environment surrounding the channel). The microchannel can be tapered at one end whereby the circumference or dimensions of the channel decrease from one end to the other so that the circumference or dimension at one end is greater than the opposing end. The microchannel of the device of the invention permits the flow of molecules, cells, small molecules or particles.

By “control” or “reference” is meant a standard of comparison. As used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence or amount of the analyte to be in question.

By “disease” is meant any condition or disorder that damages or interferes with the normal function of a cell, tissue, or organ. Examples of diseases include those associated with endothelial dysfunction.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “marker” is meant any protein or polynucleotide having an alteration in expression level or activity that is associated with a disease or disorder.

By “modulate” is meant alter (increase or decrease). Such alterations are detected by standard art known methods such as those described herein.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term, “normal amount” refers to a normal amount of a complex in an individual known not to be diagnosed with a disease or disorder. The amount of the molecule can be measured in a test sample and compared to the “normal control level,” utilizing techniques such as reference limits, discrimination limits, or risk defining thresholds to define cutoff points and abnormal values. The “normal control level” means the level of one or more proteins (or nucleic acids) or combined protein indices (or combined nucleic acid indices) typically found in a subject known not to be suffering from a disease (e.g., pulmonary hyppertension). Such normal control levels and cutoff points may vary based on whether a molecule is used alone or in a formula combining other proteins into an index. Alternatively, the normal control level can be a database of protein patterns from previously tested subjects who did not convert to a disease or disorder over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease in question or is not at risk for the disease.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

A “purified” or “biologically pure” nucleic acid or protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “reduces” is meant a negative alteration of at least 1%, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, or 100%.

By “reference” is meant a standard or control condition.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

As used herein, “obtaining” as in “obtaining an agent” includes synthesizing, purchasing, or otherwise acquiring the agent.

By “subject” is meant a mammal, including, but not limited to, a human or non-human mammal, such as a bovine, equine, canine, ovine, or feline. The subject is preferably a mammal in need of treatment, e.g., a subject that has been diagnosed with a disease or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

By “specifically binds” is meant a compound or antibody that recognizes and binds a polypeptide of the invention, but which does not substantially recognize and bind other molecules in a sample, for example, a biological sample, which naturally includes a polypeptide of the invention.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with heart disease, neurodegenerative disorders, and the like are within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The terms “treat,” “treating,” “treatment,” and the like as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

The terms “prevent”, “preventing”, “prevention”, “prophylactic treatment” and the like refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

A “therapeutically effective amount” is an amount sufficient to effect beneficial or desired results, including clinical results. An effective amount can be administered in one or more administrations.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods

and materials similar or equivalent to those described herein can be used in the practice or testing
of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1C are micrographs showing that polydimethylsiloxane (PDMS) membranes exposed to stretch followed by oxygen plasma etching produce consistent unidirectional wrinkles proportional to the direction of the strain. FIG. 1A depicts schematic illustrations of the method used to create parallel microwrinkles on a PDMS membrane using uniaxial stretch followed by plasma oxidation. FIG. 1B shows microwrinkled PDMS or control surfaces seeded with human pulmonary artery smooth muscle cells (HPASMC) at a density of 10,000 cells/cm² and cultured for 7 days. Cells on microwrinkled surfaces aligned parallel to the long axis of the wrinkles. FIG. 1C shows HPASMC cultured on the microwrinkled surfaces expressed smooth muscle alpha-actin (green). Samples were also stained for F-actin (red) and nucleus (blue).

FIG. 2 is a series of micrographs demonstrating the contractility of aligned HPASMC. HPASMC cultivated on microwrinkled PDMS were treated with angiotensin ii (1 μM) and serial phase images were taken (1, 3, 10 minutes, from left to right) to assess cell contraction. Foreshortening of the distance between the two white fiduciary marks shows the extent of cell contraction. Scale bar=50 μm.

FIG. 3A and FIG. 3B are micrographs showing that smooth muscle cells (SMCs) exhibit a contractile phenotype. Immunofluorescence labeling of smoothelin (green), F-actin (red) and nucleus (blue) in HPASMC grown on glass cover slips with adsorbed fibronectin (FIG. 3A), or type 1 collagen (FIG. 3B). Cells grown on type 1 collagen showed increased expression of smoothelin. Insets show the region of interest with 4× magnification. Scale bar=50 μm

FIG. 4 is an immunoblot showing the effects of blue light HPASMC. HPASMC were dark adapted for 1 hour followed by exposure to blue light (wave length 435 nm) for 2 minutes. SDS-PAGE of cell lysates was immunoblotted with antibodies against ppMLC, smoothelin (SMC marker) and β-tubulin. This modality may be used to verify the presence of the contractile phenotype in pulmonary vascular smooth muscle cells that are used in the model constructs that are described here (Barreto Ortiz S, et al., Am J Physiol Lung Cell Mol Physiol. 2017, PMID: 28882814).

FIG. 5A-FIG. 5C is a series of an immunoblots and graphs showing increases in arginase activity and arginase 2 protein and mRNA expression with hypoxia (a trigger for PAH). HPMEC were exposed to hypoxia (1% 02) for 24 or 48 hours. FIG. 5A shows SDS-PAGE of cell lysates were immunoblotted with antibodies against Arg 2 and tubulin. FIG. 5B shows arginase activity in HPMEC was measured using a urea assay. FIG. 5C shows Arg 2 mRNA expression by HPMEC after 0, 24 or 48 hours of hypoxia.

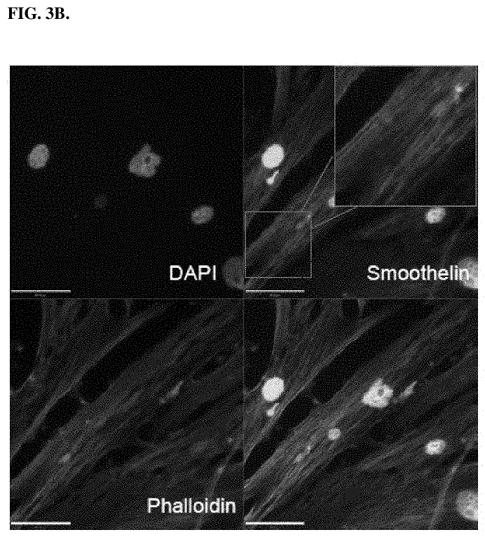

FIG. 6A and FIG. 6B is a series of photomicrographs and line graphs showing HPMEC responsiveness to uniaxial cyclic stretch. FIG. 6A shows HPMECs were subjected to mechanical stretch that recapitulated a fetal breathing pattern with 15% uniaxial stretch at 60 cycles/min (1 Hz) for 6 hours. Nitric oxide (NO) was measured at various time points (30 mins-6 hrs) and showed gradual increase in NO production with duration of stretch. FIG. 6B shows HPMEC were exposed to uniaxial stretch for 6 hr and eNOS phosphorylation and arginase 2 expression were analyzed by western blotting.

FIG. 7A and FIG. 7B show a schematic of surface modification steps, cell seeding, and film rolling. SiO/SiO₂ films are microfabricated on silicon wafer substrate and germanium (Ge) is used as the sacrificial layer. Sequential surface modification of film with (3-aminopropyl) triexthoxy silane (APTES) and glutaraldehyde enables covalent immobilization of fibronectin on the film surfaces, and this may be precisely micropatterned as shown by the stripes in FIG. 7B-FIG. 7D. Cells adhere on the modified surface, while dissolution of Ge sacrificial layer results in folding of films into tubes with cells attached on the lumen side (schematic, FIG. 7B). FIG. 7C and FIG. 7D, respectively, show an exemplary folding in rolling films and tubes, that were cultured with cells for 3 days (cells exhibit red fluorescence signal in 7D).

FIG. 8 is a diagram of the design of a biomimetic pulmonary arteriole, demonstrating the individual layered constituents. The construct is shown prior to self-assembly into a tube, and afterward.

FIG. 9A-FIG. 9C are photomicrographs showing that 3D scaffold sustained hTERT HAEC growth. FIG. 9A shows photomicrographs of rolled tubes with different dimensions seeded with cells. (L=length, OD=outer diameter). FIG. 9B and FIG. 9C shows fluorescence imaging of live cells visualized by calcein. Scale bar=200 μm.

FIG. 10A-FIG. 10F show cell viability assays of biomimetic arterioles. FIG. 10A-FIG. 10C show representative images of HPMEC (FIG. 10A), HPASMC (FIG. 10B) and arteriole EC-SMC co-culture (FIG. 10C) maintained in tubular construct evidencing live (green) and dead cells (red) at day 3. FIG. 10D-FIG. 10F show calculated cell viability as percentage of live cells using CyQUANT cell proliferation assay determining the DNA content of cells in tubular constructs, HPMEC (FIG. 10D), HPASMC (FIG. 10E) and co-culture (FIG. 10F).

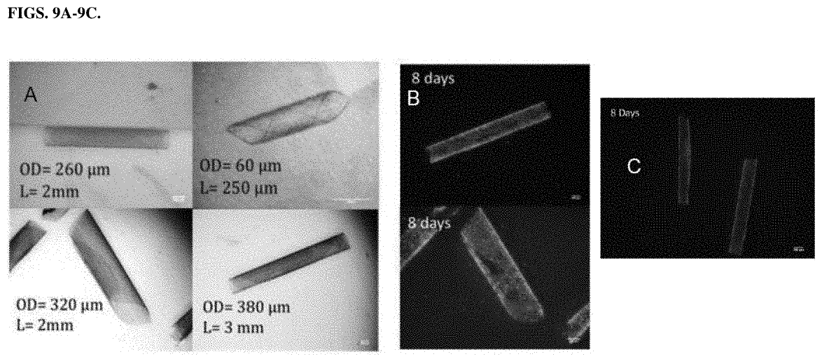

FIG. 11A-FIG. 11B show ECs and SMCs forming a monolayer in SiOx tubes. FIG. 11A is confocal microscopy images of human pulmonary microvascular endothelial cells (HPMED) showing endothelial transmembrane junction protein VE-cadherin (left), f-actin (middle) and nuclei (right). FIG. 11B is confocal microscopy images of human pulmonary artery smooth muscle cells (HPASMC) showing smooth muscle alpha actin (left), f-actin (middle) and nuclei (right).

FIG. 12A are confocal 3D images of biofabricated human pulmonary arterioles. All images were acquired using LSM800 laser scanning confocal microscope with 25× multimersion objective and 3D reconstructed using Imaris XT software. FIG. 12A is 3D reconstructed confocal images of human pulmonary microvascular endothelial cells (HPMEC) cultured on inner surface of scaffold showing endothelial cell marker VE-cadherin (green), cytoskeletal protein f-actin (red) and nuclei (blue). FIG. 12B is 3D reconstructed confocal images of human pulmonary artery smooth muscle cells (HPASMC) cultured on inner surface of scaffold showing smooth muscle marker SMαA (green), cytoskeletal marker f-actin (red) and nuclei (blue). FIG. 12C is 3D reconstructed confocal images of human pulmonary arteriole containing HPASMC and HPMEC showing cell specific markers, SMaA (red) and VE-cadherin (green) and nuclei (blue).

FIG. 13A-FIG. 13C show nitric oxide production in biomimetic arteriole. FIG. 13A is onfocal microscopy images of human pulmonary microvascular endothelial cells (HPMEC) in SiOx tube and stained with DAF-2 DA (10 μmol/L) for 30 minutes. FIG. 13A is levels of nitrite in the cell culture medium measured using Sievers Nitric oxide analyzer. Nitrite levels were normalized to total protein content on each sample. FIG. 13C is HPMECs were maintained in SiOx tubular construct or flat films for 48 hours after confluency. Cell lysates were separated by SDS-PAGE and probed with antibodies against phospho-eNOS, phospho-Akt, total eNOS and β-tubulin.

FIG. 14A and FIG. 14B show a schematic of integrated in vitro platform for testing pathobiology and therapies for pulmonary hypertension. FIG. 14A shows the modular design will allow for integration of other parts to fit the experiments. FIG. 14B shows integration of pressure-flow sensors at the ends of the arterioles to measure changes during experiment. FIG. 14A-FIG. 14B are not drawn to scale. FIG. 14C-FIG. 14D are schematic three-dimensional projections (FIG. 14C) top views, and cross-sectional views showing the device design and structure, the shape of the three tapered microchannels, and the passive trapping mechanism; (FIG. 14D, top) 3D printed mold for PDMS casting; (FIG. 14D, bottom) fabricated microfluidic platform prototype.

FIG. 15A shows an experimental setup; FIG. 15B shows trapping process including i) loading in well, ii) flowing into microchannel, iii) trapping by tapered geometry; and FIG. 15C shows flow testing in arteriolar model showing i) a bright-field image, ii) a confocal image of fluorescent particles flowing through arteriolar model with HPMEC, and iii) an optical image of fluid flow through the lumen of the model.

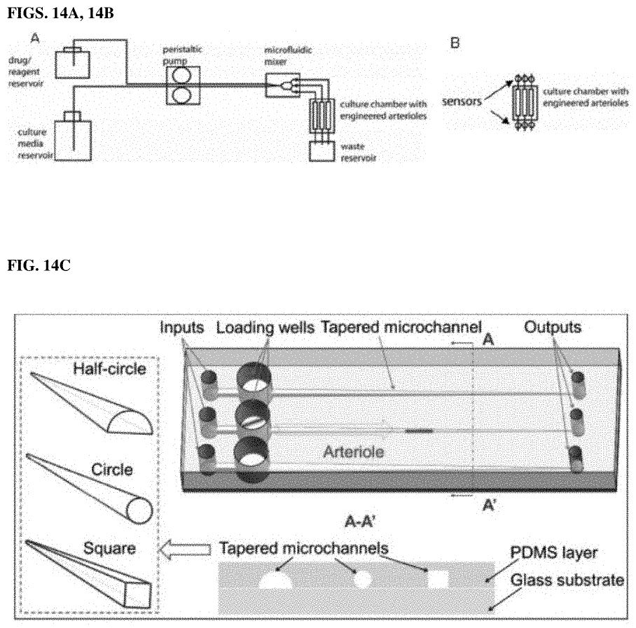

FIG. 16 is a series of micrographs depicting WI-38 cells aligned on wrinkled PDMS membranes. The left and right panels show wrinkled PDMS membranes while the middle panel shows Plain PDMS.

FIG. 17A is a perspective view of EC tube in 3D. Confocal microscopy images of human pulmonary microvascular endothelial cells (HPMEC) showing endothelial transmembrane junction protein VE-cadherin (green), f-actin (red) and nuclei (blue) with scale bar 200 μm.

FIG. 17B-FIG. 17C show micro-patterning of smooth muscle cells (HPASMC). FIG. 17B is i) 3D rendered confocal image of HPASMC grown on scaffolds with patterend fibronectin induced cell alignment that mimic in vivo SMC orientation. FIG. 17C is i) without patterning cell attached and spread in random orientation. Fiber orientation analyses suggests that overall F-actin are more parallel in patterned fibronectin (FIG. 17B, ii) compared to unpatterned fibronectin (FIG. 17C, ii). Polar plot of the angle of orientation of Factin highlights the wider spread of fiber orienatation in cells grown on unpatterned fibronectin (FIG. 17C, iii) compared to pattered fibronectin (FIG. 17B, iii) Binning in polar plots is 10°.

FIG. 18 is a schematic demonstrating the epidemiology of pulmonary hypertension during both development and aging.

FIG. 19 is an exemplary photograph depicting the key relationship in pulmonary hypertension between endothelium and smooth muscle cell as described in related arts (Collaco, Romer, et al., Peds Pulmonol, 2012, doi: 10.1002/ppul.22609; Waruingi and Mhanna, World J Pediatr, 2014, doi: 10.1007/s12519-014-0464-2; Adir and Amir, Semin Respir Crit Care Med, 2013, doi: 10.1055/s-0033-1356490).

FIG. 20 is a schematic representation of an embodiment of a microfluidics-based model of the pulmonary circulation system.

FIGS. 21A-21F is a schematic representation of a fabrication process of the microfluidic device. FIG. 21A: A 3D-printed mold is used to build a 3D topographic microchannel which is not accessible by conventional lithography; FIG. 21B: PDMS (1:10 w/w curing agent to prepolymer ratio) is cast on top of the 3D printed mold (the mold was pre-treated with HMDS (hexamethyldisilazane) to facilitate demolding; FIG. 21C: demolding the PDMS device and puncturing opening ports; FIG. 21D: a 25-μm thick PDMS slab is deposited on a clean glass slide to serve as the bottom layer of the microchannel; FIG. 21E: after O₂ plasma activation, top and bottom PDMS pieces are bonded together and thermally cured. FIG. 21F shows the fabricated prototype.

FIG. 22 is a schematic representation of an experimental setup for flow circulation and hydrostatic pressure measurements.

FIGS. 23A-23C are a schematic representation of passive trapping of a 2 mm-long biomimetic arteriole in the tapered microchannel: Images demonstrate the arteriole FIG. 23A in the loading well, FIG. 23B flowing into the microchannel, and FIG. 23C trapping by the tapered channel geometry.

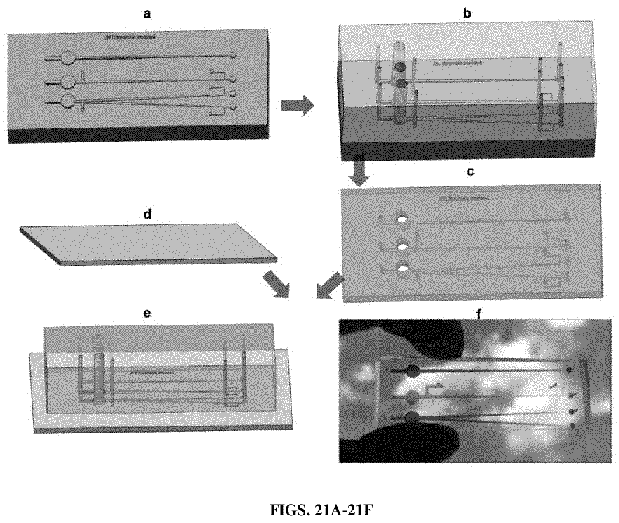

FIG. 24 is a scan of a photograph showing an epifluorescence imaging setup: Flow test of fluorescent microparticles through a 3D biomimetic arteriole (length 2 mm long, inner diameter 380 μm) that is inside the microchannel.

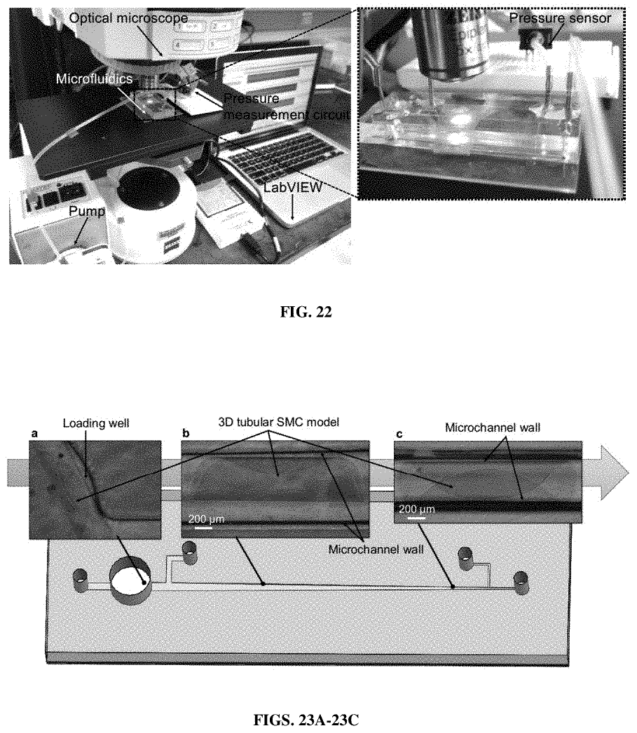

FIG. 25 is a scan of a photograph showing a custom-designed graphical user interface: Real-time pressure waveforms at the upstream and downstream monitoring positions are shown; the difference between them is displayed in real time. The pressure ranges and pulse frequency generated in the microfluidic device are close to those seen in the human pulmonary circulation. The tapered microchannel recapitulates the arteriolar resistance component of the pulmonary circulation that regulates flow by pulmonary vascular resistance.

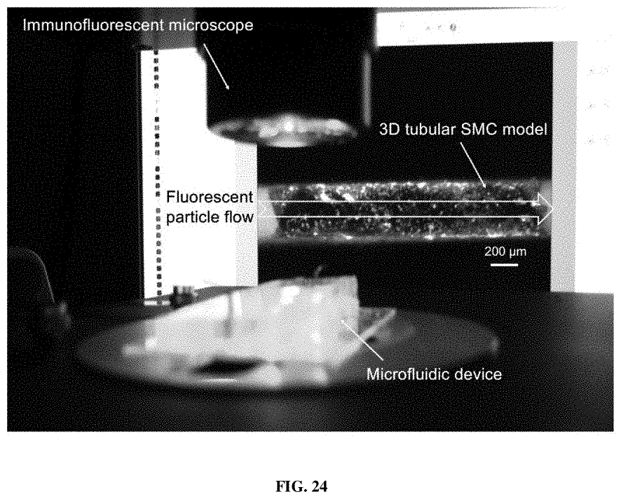

FIGS. 26A, 26B are graphs showing: FIG. 26A: Pressure waveforms from the upstream port of the microchannel at flow rates of 0.11, 0.13, and 0.15 mL/min, respectively; FIG. 26B: the relationship of systolic pressure to flow rate in this microfluidics platform.

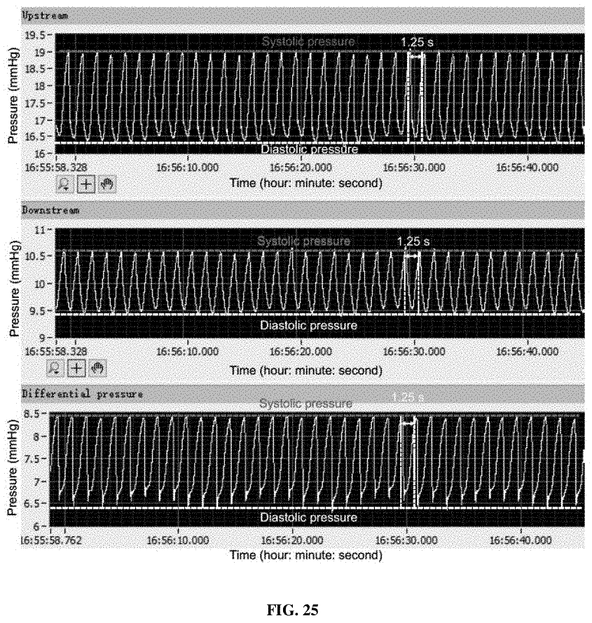

FIG. 27 is a plot of mean pressure difference between the upstream and downstream ports of the microchannel that are measured at three flow rates. The vascular resistance shows pulse frequency dependence, suggesting that it is a more complex impedance model that involves microchannel compliance as well as resistance.

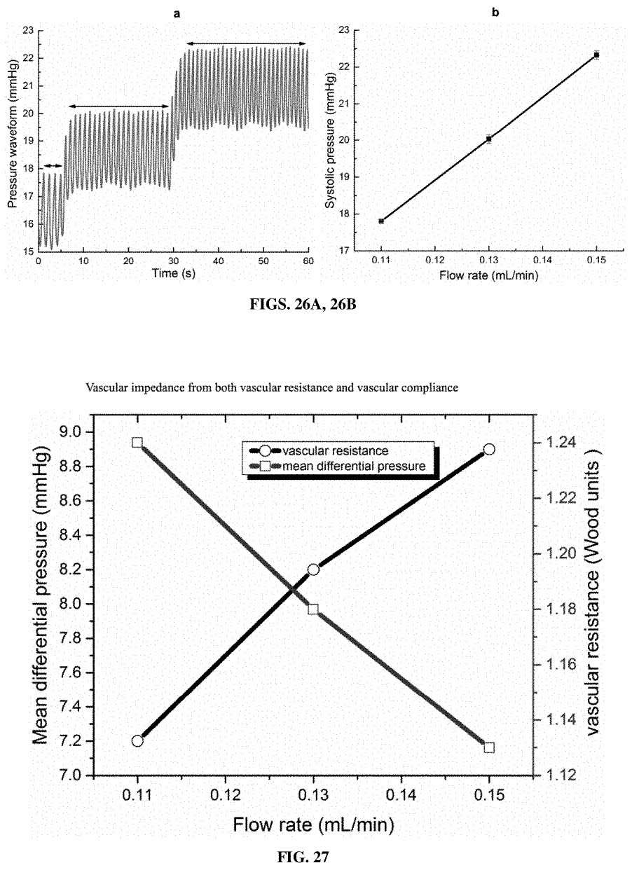

### DETAILED DESCRIPTION

The invention is based, at least in part, on the surprising discovery that biomimetic platforms and tissues can be developed for studying pulmonary hypertension. Described herein are compositions of human pulmonary artery smooth muscle cells (HPASMC) cultivated on micro-wrinkled elastomeric polydimethylsiloxane (PDMS) sheets to mimic pulmonary arteriole structure and function. Furthermore, the invention also provides for a platform that mimics the 3D architecture and function of human pulmonary arterioles. Described herein are compositions used to examine the pathobiology of as well as potential therapies to pulmonary hypertension.

### Overview of Human Pulmonary Artery Smooth Muscle Cells (HPASMC) for Studying Pulmonary Arterial Hypertension (PAH)

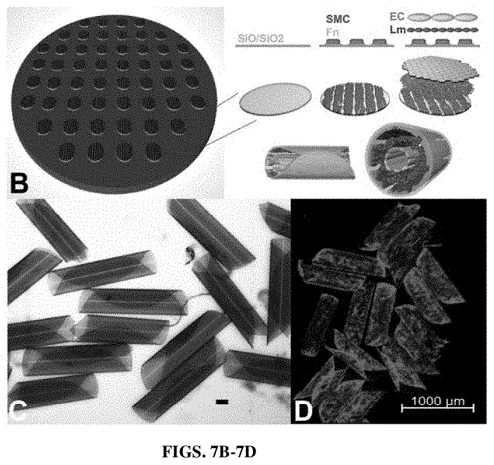

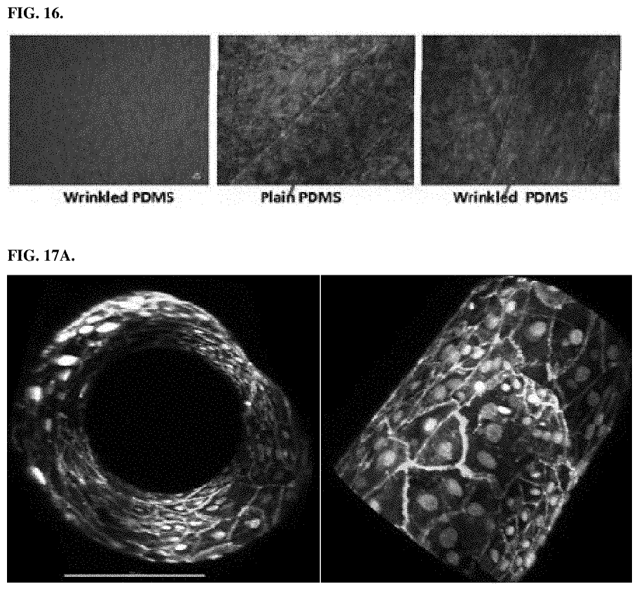

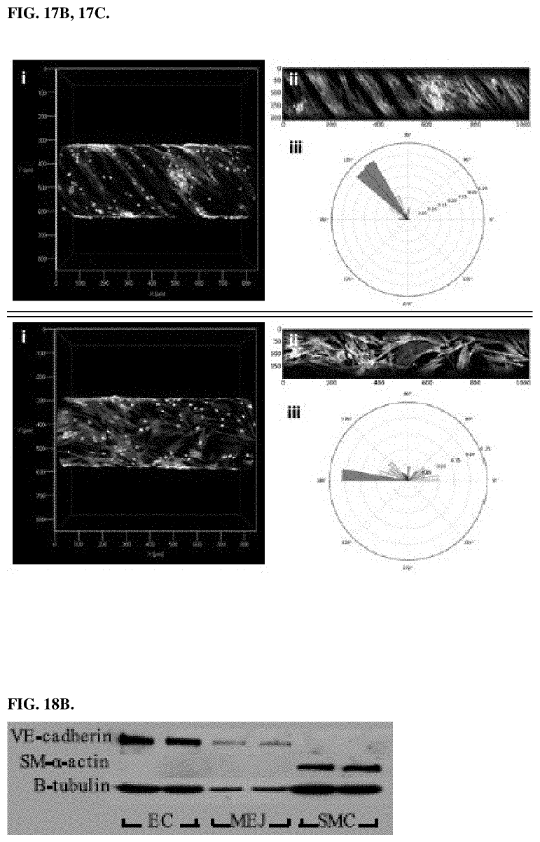

Pulmonary arterial hypertension (PAH) is a progressive and often fatal illness of the pulmonary microvasculature (Galie, N., Pulmonary Arterial Hypertension: Epidemiology, Pathobiology, Assessment, and Therapy, 2004. Elsevier; Farber H. W., and Loscalzo J., New England Journal of Medicine, 2004. 351:1655-1665). The prevalence of PAH in the US has been estimated at 109 per million among individuals under 65, and 451 per million among those 65 and over, with average survival after diagnosis estimated at 5 to 7 years (Benza, R. L., et al., Chest, 2012. 142:448-56; Kirson, N.Y., et al., Curr Med Res Opin, 2011. 27:1763-8) (see FIG. 18). Fundamental questions regarding mechanisms underlying the pathogenesis of PAH still remain Animal models of PAH have limitations, as they differ from humans lungs with regard to the anatomy of the pulmonary circulation (Voelkel N. F., and Tuder R. M., J Clin Invest, 2000. 106:733-8; Campian, M. E., et al., Naunyn SchmiedebergsArch Pharmacol, 2006. 373:391-400). To address this gap, the development of a biomimetic in vitro model system that accurately recapitulates key elements of the human pulmonary arteriole, such as cellular constituents and morphology, tissue infrastructure, and cell-cell communication has been proposed, as described herein. Despite remarkable advancements in the field of blood vessel bioengineering, no such structural and functional prototype exists. Pulmonary arterioles, comprised primarily of endothelial (EC) and smooth muscle cells (SMC), are a unique subset of blood vessels within the pulmonary circulation that are responsible for controlling blood flow (see FIG. 8). This requires communication across the inner elastic lamina (IEL), and formation of finely tuned heterotypic adhesions —termed myoendothelial junctions (MEJ) (Isakson B. E., and Duling B. R., CircRes, 2005. 97:44-51; Isakson, B. E., et al., Circ Res, 2007. 100:246-54; Heberlein, K. R., et al., Circ Res, 2010. 106:1092¬102; Straub, A. C., et al., Nature, 2012. 491:473-7) (see FIG. 19). As described herein, this disclosure focuses on reconstituting and studying this highly specialized communication link in a precisely engineered elastomeric platform.

The prevalence of PAH in the US has been estimated at 0.003%-0.005% of the total population. Right ventricular failure is the main cause of death in patients with PAH with a low post-diagnosis survival estimated between 5 to 7 years (Chin, K. M., et al., Coron Artery Dis, 2005. 16:13-18; Sitbon, O., et al., Am JRespir Crit Care Med, 2008. 177:108-13). In patients with PAH, the right ventricle (RV) is exposed to pressure overload due to increased resistance to flow in pulmonary arterioles, resulting in RV dilatation, hypertrophy, and failure (Simonneau, G., et al., JAm Coll Cardiol, 2009. 54:S43-54). Hence, pulmonary arterioles represent an attractive research target. Currently, PAH has no effective long term treatment, and lung transplantation is reserved for patients with end-stage disease (Chin K. M., and Rubin L. J., JAm Coll Cardiol, 2008. 51:1527-38; Huddleston, C. B., Pediatr Crit Care Med, 2010. 11:S53-6). Independent of its etiology, PAH is a cardiovascular disease associated with augmented vasoconstriction, inflammation, an imbalance of vasoactive mediators, and vascular remodeling (Allen K. M., and Haworth S. G., JPathol, 1989. 158:311-7; Humbert, M. et al., JAm Coll Cardiol, 2004. 43:13S-24S; Steinhorn R. H., and Fineman J R, Artif Organs, 1999. 23:970-4). Understanding pathophysiological mechanisms that cause PAH is key to designing effective therapies and reducing the health care cost burden associated with this disease. One of the barriers to these goals is the lack of an animal model that adequately recapitulates the complex structure, microenvironment and physiological functionality of living human lung (Stenmark, K. R., et al., Am JPhysiol Lung Cell Mol Physiol, 2009. 297:L1013-32). There are marked differences between human lung vascular architecture and that found in other species. These include the multiple intersections between the bronchial and pulmonary microcirculations in the mouse lung and the overall vascular layout and branching pattern of vascular networks (McLaughlin, R. F., et al., Med Thorac, 1962. 19:523-7). These anatomical differences alter both physiology and pathophysiologic responses, and interfere with direct translation of the results of animal model experiments into clinical research (McLaughlin, R. F., et al., Med Thorac, 1962. 19:523-7).

Human Biomimetic Blood vessels

Despite the availability of numerous bioengineered model vessels, the construction of an entirely biomimetic blood vessels has not yet been accomplished. From a biological standpoint, this type of vessel construct should not only consist of a vascular tube with cellular elements inherent to blood vessels, but should also replicate the endogenous extracellular matrix which allows for sustained maintenance of resident cells. Some of the more successful synthetic vessels in the field are tissue-engineered vessel grafts (TEVG) (Weinberg C. B., and Bell E., Science, 1986. 231:397-400). Despite commercial potential of TEVG in the vascular device market, there have been numerous limitations: fabrication techniques limit them to large vessel models; they lack microenvironmental features of arterioles; they are prone to inflammation, thrombosis, and rejection; and their mechanical properties do not replicate in vivo conditions (L'Heureux, N., et al., Nat Med, 2006. 12:361-5). Scaffold-free blood vessel approaches through which EC and SMC synthesize their own matrix scaffolds are underway, but they do not recapitulate functional gap junctions between cell layers (Jung, Y., et al., Sci Rep, 2015. 5:15116; Kelm, J. M., et al., J Biotechnol, 2010. 148:46-55). More recently, microdevices known as arteries-on-a-chip mimic some of the physiological functionality of living human organs, but these lack the mechanics of natural blood vessels and communication between EC and SMC via an inner elastic lamina (Yasotharan, S., et al., Lab Chip, 2015. 15:2660-9). As described herein, the in vitro model offers the possibility of overcoming most of these limitations. Firstly, the construct is analogous to a pulmonary arteriole in regards to its cellular constituents. Secondly, SMC population is maintained in a contractile phenotype, with anatomically relevant parallel alignment that is facilitated by incorporating wrinkles on the elastomer surface using lithography. Lastly, and most importantly, the elastomer construct is microfabricated to facilitate functional gap junction formation between the EC and SMC. Taken together, the elastomeric construct described herein provides a biologically relevant in vitro model to study PAH. Moreover, it facilitates the study of PAH related specific signaling changes at the heterocellular junctions between pulmonary EC and SMC.

### Myoendothelial Junctions, Nitric Oxide, and Arginase

Intercellular communication between cells within the blood vessel wall plays an important role in vessel function (Dora, K. A., Vasc Med, 2001. 6:43-50). Specialized cellular extensions from endothelium and smooth muscle cells protrude through the inner elastic lamina where they interact with the opposing cell type. These structures, termed myoendothelial junctions (MEJ), have been cited as key elements in vascular physiology and disease (Straub, A. C., et al., Arterioscler Thromb Vasc Biol, 2011. 31:399-407). These gap junctions allow the transfer of second messengers and electrical signaling between the two cell types, thus synchronizing their function. One of the signaling molecules that is transported across the MEJ is nitric oxide (NO), a potent endothelium-derived relaxing factor. NO induces smooth muscle relaxation via the guanylate cyclase system, leading to increased cGMP levels (Murad, F., J Clin Invest, 1986. 78:1-5). It has been demonstrated that eNOS is compartmentalized at the MEJ and that local regulation of eNOS/NO pathway may occur at the MEJ (Straub, A. C., et al., Arterioscler Thromb Vasc Biol, 2011. 31:399-407). In vascular endothelium, NO is produced by eNOS which utilizes L-arginine as a substrate. Arginase 2 is also expressed in the endothelium, and reciprocally regulates nitric oxide (NO) levels in endothelial cells by competing with eNOS for the common substrate L-arginine. Hence, L-arginine bioavailability is crucial for adequate production of NO by the endothelium. Arginase 2 has been implicated in many vasculopathies (Pandey, D., et al., Circ Res, 2014. 115:450-9; Berkowitz, D. E., et al., Circulation, 2003. 108:2000-6; Xu, W., et al., FASEBJ, 2004. 18:1746-8). As described herein, arginase 2 was recently identified as a key modulator in oxidized-LDL-mediated atherogenic vasculopathy. Arginase relocates of 2 from mitochondria to the cytosol after oxidative injury, and competes with eNOS for cytosolic L-arginine (Pandey, D., et al., Circ Res, 2014. 115:450-9). The focus of the experiments, as described herein, is the MEJ as a subcellular domain for eNOS/arginase signaling and a possible target for therapies that limit arginase expression and/or activity in PAH. Studies of the MEJ have been heretofore conducted using either non-human or non-lung cells. As described herein, NO regulation at MEJ between human lung microvascular EC and SMC is examined.

Study of signaling events involved in PAH has been limited to animal and cell culture models. A significant limitation of the animal models is insufficient similarity to human lung structure and physiology. In vitro studies with cell culture models lack multi-layered cell populations and interactions between endothelium and smooth muscle cells. As described herein, these shortcomings are addressed by building an in vitro construct that recapitulates both structural and functional features of the in vivo lung microvessel. The functionality of MEJ between EC and SMC from human pulmonary arteriole is investigated on the elastomeric platform described herein. Moreover, nitric oxide signaling, known to be downregulated in PAH, is studied at the MEJ to decipher the role of the critical NO signaling regulators, arginase 2 and eNOS.

As described herein and in the Examples below, human pulmonary artery smooth muscle cells (HPASMC) have been cultivated on micro-wrinkled elastomeric polydimethylsiloxane (PDMS) sheets, which resulted in alignment of HPASMC along the wrinkles. The use of photolithography, interference lithography, and nano-imprint lithography have been investigated to create ordered ridges (topography) with high precision and tunability to control cellular alignment. SMC contraction and relaxation are assessed by measuring changes in spacing between microfabricated troughs in the PDMS membrane that are perpendicular to the axis of HPASMC alignment.

Commercially available transwell co-culture inserts were used to study formation of functional MEJ between HPASMC and HPMEC. MEJ is characterized by western blotting and confocal imaging of cell type-specific protein constituents, and by scanning electron microscopy. The elastomeric model described herein is used to further characterize the function of human lung MEJ. The effects of hypoxia and arginase 2 on nitric oxide signaling through these MEJ are studied. Hypoxia upregulates arginase 2 in HPMEC, thereby limiting EC nitric oxide (NO) production and hampering HPASMC relaxation.

As described herein, material science, tissue engineering, and vascular and molecular biology are incorporated to fine tune an in vitro elastomeric platform that constitutes key elements of the pulmonary microvessel—human EC and SMC with physiologically relevant morphology, alignment, and functional heterotypic connections through MEJ. The structure, cellular composition, and versatility of this platform offers opportunities to address complex and pressing questions regarding PAH that cannot be studied with current approaches. These include high throughput screening of candidate therapies for PAH that accelerates the drug development process.

Blood pressure and tissue perfusion are regulated in part by resistance arteries, comprised primarily of endothelial and smooth muscle cells. Described herein are components of a three-dimensional biomimetic blood vessel model that includes human pulmonary artery smooth muscle cells (SMC) and a monolayer of pulmonary microvascular endothelial cells (EC). Alignment of smooth muscle cells in a circumferential axis is key in ensuring proper vessel contraction and relaxation. As a first approach to alignment, a polydimethylsiloxane (PDMS) membrane was fabricated with microwrinkles on the surface. SMCs cultivated on this micro-wrinkled surface aligned perpendicular to the long axis of the vessel, in contrast with those grown on plain PDMS surfaces. Moreover, SMC that were aligned in this geometry exhibited increased expression of smooth muscle contractile proteins including smooth muscle alpha-actin. Creation of a functional inner elastic lamina (IEL) separating the SMC and EC was also demonstrated. Toward this end, type I collagen and laminin were studied with regard to development of the contractile phenotype in the SMC. Furthermore, the formation of myoendothelial junctions (MEJ) within the IEL is essential for communication between EC and SMC. Using a vascular cell co-culture model, MEJ formation between these two cell types was demonstrated, useful for defining the constituents and signaling events that characterize these junctions in the human pulmonary microvasculature.

Surface modification with microwrinkles and type-1-collagen coating favors HPASMCs towards a contractile phenotype, an important characteristic of vascular smooth muscle cells in vivo. Transwell inserts used to successfully form MES is a co-culture to study important signaling molecules that is further incorporated into the biomimetic blood vessel. Cyclic stretch mimicking fetal breathing parameters induces eNOS phosphorylation and robust increase in NO production. In the long term, this is used as a potential readout for functionality of the biomimetic vessel.

### Overview of 3D Biomimetic Platforms for Studying Pulmonary Arterial Hypertension

Pulmonary hypertension is a debilitating disorder caused by congenital or acquired diseases of the heart or lungs. Vasoconstriction and structural remodeling of arterioles are principal events in the disease pathogenesis (Tuder, R. M., et al., Clin Chest Med, 2007. 28 (1), 23-42, vii). There are no known cures for this condition at this time, and it has a reported mortality rate of up to 48% (Barst, R. J., Ann Thorac Med, 2008. 3 (1), 1-4). Most of the drugs used in managing pulmonary hypertension are based upon molecular signaling between endothelial cells and smooth muscle cells (Rich S., and McLaughlin V V., 2003. 108 (18), 2184-90; Makowski, C. T., et al., Pharmacotherapy 2015. 35 (5), 502-19). It is therefore important to better understand how these cells communicate and regulate pertinent molecular signaling pathways that affect their function and morphology. Traditionally, flat cell culture platforms are used to investigate how cells behave, but these methods lack 3D structural features and the resulting biology that is present in vivo (Ravi, M., et al., JCell Physiol, 2015. 230 (1), 16-26; Baker B. M., and Chen C. S., JCell Sci, 2012. 125 (Pt 13), 3015-24). Animal models used in in vivo studies are expensive and sometimes produced different effects than those seen in humans (Ameen, N., et al., Histochem Cell Biol, 2000. 114 (1), 69-75). Therefore, as described herein, a focus of this invention is to develop a 3D in vitro platform that recapitulates the in vivo architecture and function of human pulmonary arterioles.

By developing a platform that mimics the 3D architecture of pulmonary arterioles and includes the capability to modulate mechanical forces such as shear stress and cyclic stretch, the behavior of native tissue is more accurately mimicked. The 3D platform consists of self-folding, thin bilayer films of silicon oxide and silicon dioxide. This approach is scalable and highly reproducible using conventional lithographic patterning and strain engineering (Xi, W., et al., Nano Letters, 2014. 14 (8), 4197-4204). As compared to prior self-folding approaches, these techniques do not require any harsh chemical treatments during release and self-rolling of the film (Barreto-Ortiz, S. F., et al., FASEB J, 2015. 29 (8), 3302-14; Sigusch, B. W., et al., Dent Mater, 2014. 30 (6), 661-8; Randall, C. L., et al., Lab Chip, 2011. 11 (1), 127-31). Cells are seeded while the films are still flat and the structures roll-up with precisely engineered time frames and luminal diameters. Further, integrating this microvascular model into microfluidic systems enables examination of the effects of flow on mechanochemical signaling (Myers, D. R., et al., J VisExp, 2012. (64); Tsai, M., et al., J Clin Invest, 2012. 122 (1),408-18). As described herein, a precisely engineered, tunable, functional, and anatomically accurate biomimetic human pulmonary microvasculature that supports quantitative analysis of endothelial and smooth muscle cell communication in pulmonary hypertension was developed.

The pathogenesis of pulmonary vascular remodeling resulting in decrease of vessel elasticity and of inner diameter is still not fully understood (Hu, J., et al., Am J Cardiovasc Drugs, 2015. 15 (4), 225-34). Therefore, there is a pressing need to hasten understanding of this disease. An in vitro system that mimics in vivo architecture, chemical, and mechanical cues is beneficial in accelerating the discovery of potential therapeutic targets and understanding of the pathobiology of pulmonary hypertension. Tissue engineering of large arteries has been previously demonstrated using decellularized scaffolds, molds, and cell-sheet, (Weinberg C. B., and Bell E., Science, 1986. 231 (4736), 397-400; L′Heureux, N., et al., FASEBJ, 1998. 12 (1),47-56) but there exists a great need to develop tissue-engineered microvasculatures such as arterioles, resistance vessels important in the pathology of cardiovascular diseases (Barreto-Ortiz, S. F., et al., FASEB J, 2015. 29 (8), 3302-14). Arterioles vary in diameter sizes but the distinguishing feature is the presence of one or two layers of smooth muscle cells in the wall of the vessel surrounding the endothelial cells (Martinez-Lemus, L. A., Basic Clin Pharmacol Toxicol 2012. 110 (1),5-11). Using mold and manual rolling make it difficult, if not impossible, to produce delicate structures such as arterioles in a reproducible and high-throughput manner.

Microfabrication is a high-throughput method developed initially for microchip manufacturing that follows a sequence of steps to produce reproducible structures in micrometer to nanometer dimensions. Microfabrication technology has been adapted from the electronic industry to develop miniature devices composed of biologically relevant materials (Pagaduan, J. V., et al., Anal Bioanal Chem, 2015. 407 (23), 6911-22). This technology has also been utilized to produce functional self-folding films such as tether-less microgrippers, (Malachowski, K., et al., Nano Lett, 2014. 14 (7), 4164-70; Malachowski, K., et al., Angew Chem Int Ed Engl, 2014. 53 (31), 8045-9; Gultepe, E., et al., Gastroenterology, 2013. 144 (4), 691-3) and 3D structures for medical applications (Park, J., et al., Artif Organs, 2013. 37 (12), 1059-67). 3D microtubes made up of a biodegradable strain-engineered silicon oxide bilayer have been demonstrated to be applicable for tissue culture. Previous reports were able to roll up tubes, but due to limitations in the process such as the use of toxic chemical etchants, it was only possible to seed the cells after the films folded into tubes (Arayanarakool, R., et al., Lab Chip, 2015. 15 (14), 2981-9). Hence, similar to other methods of seeding cells on tubular fibers, it is challenging to deposit cells uniformly around the tube as well as layer cells. A key innovation, as described herein, has resulted in a process where cells are deposited and layered uniformly on flat sheets prior to roll-up. This is achieved by using sacrificial layer that dissolves in culture media. For the first time it is possible to create precisely layered co-cultures in a tubular geometry.

Cell co-culture is a step towards mimicking in vivo environment. Co-culture of cells in transwells where two different cell types are cultured on opposite sides of a membrane has shed light on paracrine signaling and the importance of myoendothelial junction in endothelial and smooth muscle cell communications (Gairhe, S., et al., Am J Physiol Lung Cell Mol Physiol, 2011. 301 (4), L527-35). Cell sheet technology has enabled direct contact co-culture by harvesting cell sheet from thermoresponsive surface and layering different cell sheets that closely resemble in vivo environment but the production of intima may take longer to mature (Truskey, G. A., Int J High Throughput Screen, 2010. (1), 171-181). Though useful in elucidating important cell-to-cell signaling, both methods lack geometric orientation present in vivo. The self-folding scaffold described herein allows spatial orientation of cells and 3D geometry found in vivo. Aside from spatial and geometric organization, chemical cues are important for cell growth and function.

Extracellular matrix proteins and proteoglycans are used in the scaffold described herein using silane surface modifications. It is important to select the appropriate type of extracellular matrix for cell culture. Several extracellular matrix (ECM) proteins such as fibronectin, laminin and collagen have been studied to promote cell adhesion and proliferation (Thyberg, J. and Hultgardh-Nilsson, A., Cell Tissue Res 1994, 276 (2), 263-71). Fibronectin has been used to promote endothelial cell adhesion and proliferation (Wijelath, E. S., et al., J Vasc Surg 2004, 39 (3), 655-60). Synthetic phenotypes were observed when smooth muscle cells were grown on fibronectin-coated surfaces (Yamamoto, M., et al., Exp Cell Res 1993, 204 (1), 121-9). On the other hand, laminin maintained contractile phenotype for smooth muscle cells (Qin, H., et al., Exp Mol Pathol 2000, 69 (2), 79-90; Alford, P. W., et al., Integr Biol (Camb) 2011, 3 (11), 1063-70). Others have also shown that the co-culture of cells promotes ECM deposition. For example, co-culture of pericytes with endothelial cells dramatically increased ECM deposition of endothelial cells in vitro compared to endothelial cells alone (Stratman, A. N., et al., Blood 2009, 114 (24), 5091-101). ECM also directly affects the signal transduction within the cells depending on which integrins are activated. Studies have shown that 3D and 2D cell cultures have different effects on cell behavior, gene expression, morphology, and function (Ravi, M., JCell Physiol 2015, 230 (1), 16-26). Furthermore, cell cultures in traditional 2D flat cell culture flasks have been observed to have different drug response compared to cells in 3D systems due to unnatural microenvironement (Edmondson, R., et al., Assay Drug Dev Technol 2014, 12 (4), 207-18).

Hemodynamic forces, stimuli absent in 2D static cultures, such as shear stress and cyclic stretch induce mechanotransduction (Polacheck, W. J., et al., Proc Natl Acad Sci USA2014, 111 (7), 2447-52; Zheng, W., et al., Am J Physiol Heart Circ Physiol 2008, 295 (2), H794-800). Microfluidics have been used to integrate flow and cyclic stretch on cell culture to mimic physiological mechanical cues (Huh, D., et al., Science 2010, 328 (5986), 1662-8). It has been shown that endothelial cells subjected to cyclic stretch, exhibit increased orientation or anisotropy (Wang, J. H., et al., J Biomech 2001, 34 (12), 1563-72) and increase endothelial nitric oxide synthase expression (Toda, M., et al. J Biotechnol 2008, 133 (2), 239-44). As described herein, the complete system integrates a microfluidic system with the capability to measure in situ changes in flow, pressure, and resistance, while also integrating live-cell imaging by microscopy.

Integration of engineering, biology and chemistry allowed demonstration of tissue-engineering arterioles using biodegradable, self-folding scaffolds with tunable dimensions. Even cell distribution and cell layering on flat sheets prior to roll up is possible for the first time. The surface of the scaffolds is easily modified to suit any cell type to be studied. Another key innovation, as described herein, is the use of microfluidics to induce mechanical cues that completes the recapitulation of in vivo environment. This complete system allows parallel analysis of multiple arterioles in a single, integrated platform with an automated drug delivery system and pressure and flow sensors.

A variety of materials and methods, according to certain aspects of the invention, can be used to form any of the described components of the systems and devices of the invention. In certain embodiments, the device is produced by use of 3D printing methods. In other example, the various materials selected lend themselves to various methods. For example, various components of the invention can be formed from solid materials, in which the channels can be formed via molding, micromachining, film deposition processes such as spin coating and chemical vapor deposition, laser fabrication, photolithographic techniques, etching methods including wet chemical or plasma processes, and the like. At least a portion of the fluidic system can be formed of silicone by molding a silicone chip. Technologies for precise and efficient formation of various fluidic systems and devices of the invention from silicone are known. Various components of the systems and devices of the invention can also be formed of a polymer, for example, an elastomeric polymer such as polydimethylsiloxane (“PDMS”), polytetrafluoroethylene (“PTFE”) or TEFLON™, or the like.

The channels of the invention can be formed, for example by etching a silicon chip using conventional photolithography techniques, or using a micromachining technology called “soft lithography”. These and other methods may be used to provide inexpensive miniaturized devices, and in the case of soft lithography, can provide robust devices having beneficial properties such as improved flexibility, stability, and mechanical strength. When optical detection is employed, the invention also provides minimal light scatter from molecule, cell, small molecule or particle suspension and chamber material.

Different components can be formed of different materials. For example, a base portion including a bottom wall and side walls can be formed from an opaque material such as silicone or PDMS, and a top portion can be formed from a transparent or at least partially transparent material, such as glass or a transparent polymer, for observation and/or control of the fluidic process. Components can be coated so as to expose a desired chemical functionality to fluids that contact interior channel walls, where the base supporting material does not have a precise, desired functionality. For example, components can be formed as illustrated, with interior channel walls coated with another material. Material used to form various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

Various components of the invention when formed from polymeric and/or flexible and/or elastomeric materials, and can be conveniently formed of a hardenable fluid, facilitating formation via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are preferred, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying formation of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures of the invention from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus components can be formed and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.).

To prevent material (e.g., cells and other particles or molecules) from adhering to the sides of the microchannels, the microchannels may have a coating which minimizes adhesion. Such a coating may be intrinsic to the material from which the device is manufactured, or it may be applied after the structural aspects of the microchannels have been microfabricated. Polytetrafluoroethylene (TEFLON™) is an example of a coating that has suitable surface properties. The surface of the microchannels of the microfluidic device can be coated with any anti-wetting or blocking agent for the dispersed phase. The microchannel can be coated with any protein to prevent adhesion of the biological/chemical sample. For example, in one embodiment the microchannels are coated with BSA, PEG-silane and/or fluorosilane. For example, 5 mg/ml BSA is sufficient to prevent attachment and prevent clogging. In another embodiment, the microchannels can be coated with a cyclized transparent optical polymer obtained by copolymerization of perfluoro (alkenyl vinyl ethers), such as the type sold by Asahi Glass Co.

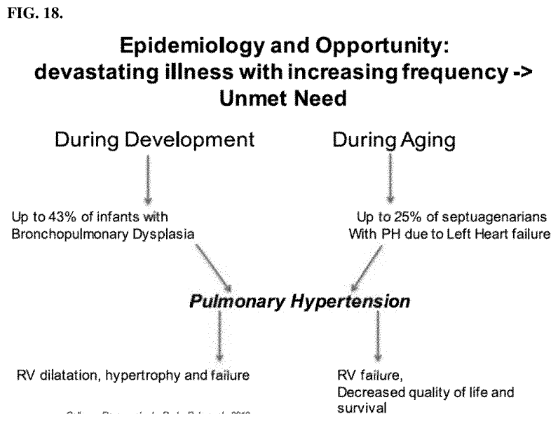

The microfluidic devices embodied herein can be designed to model various in vivo human or animal systems. In certain embodiments, the microfluidic device is designed to recapitulate the pulmonary microvasculature with respect to tapered morphology, materials compliance, physiologically relevant pulsatile flow pattern and flow rate, and range of pulmonary blood pressure (FIG. 20). The input and output ports at two ends of the channel are used for circulation of flow. The loading well is used to introduce 3D model vessels for focused study. Pressure readout ports at the upstream and downstream ends of the channel are used to track systolic and diastolic pressures in real-time. The pressure difference is used to interrogate the vascular resistance across the microchannel.

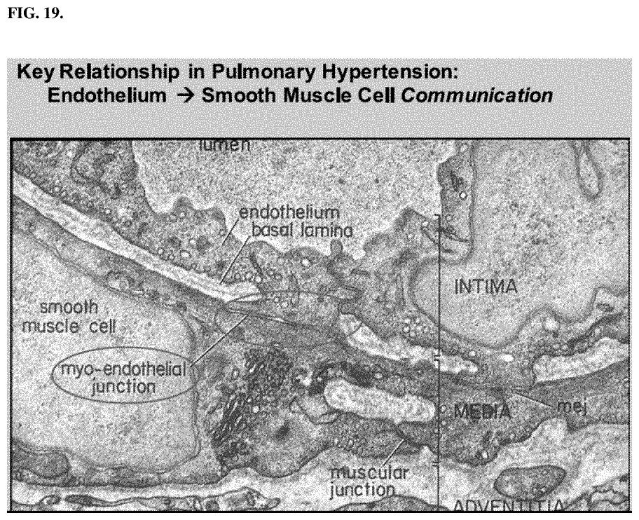

In certain embodiments, a 3D-printed mold is used to build a 3D topographic microchannel which is not accessible by conventional lithography (FIGS. 21A-21F). In certain embodiments, the PDMS (for example, 1:10 w/w curing agent to prepolymer ratio) is cast on top of the 3D printed mold (the mold can be pre-treated with HMDS (hexamethyldisilazane) to facilitate demolding. The PDMS device is demolded and opening ports are punctured. In certain embodiments, a PDMS slab having a thickness from about 0.1 μm to about 50 μm is deposited on a clean glass slide to serve as the bottom layer of the microchannel After oxygen (O₂) plasma activation, the top and bottom PDMS pieces are bonded together and thermally cured.

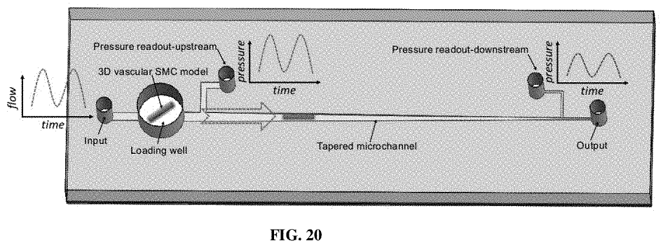

An experimental setup for flow circulation and hydrostatic pressure measurement is shown in FIG. 22. In this embodiment, a peristatic pump is used to supply tunable flow patterns to the microchannel Two piezoresistive pressure sensors connected to two branches of ports can transduce hydrostatic pressure to an electrical resistance signal, and then convert it to digital data for signal processing using the LabVIEW data acquisition system. Bright-field imaging of the interior of the microchannel may be done via an optical microscope above the microfluidics device.

TABLE 1
Comparison between mechanics of the human pulmonary circulation
and the microfluidics model developed herein.

| | | Micro- |
|---|---|---|
| | Pulmonary circulation | fluidic |
| Parameters | (adult, unless otherwise specified) | model |
| Blood | Normal: 8-20 (rest) −> 30 | 11-60 |
| pressure | (with exercise) | |
| (mmHg) | Hypertension: > 8-20 (rest) −> 30 | |
| | (with exercise) | |
| Blood flow | Total cardiac output: 3000-8000 | 0.1-0.15 |
| (mL/min) | (adult) | |
| | One single arteriole | |
| | (200 μm in diameter): 0.08-0.15 | |
| | (calculated from estimated number | |
| | of arterioles) | |
| Blood | 1 (adult) | 0.8-1.1 |
| frequency | | |
| (Heart Rate) | | |
| (Hz) | | |
| Pulmonary | Total vasculature resistance: | 1-5.7 |
| resistance | 0.25-3.0 | 19-108 × 10⁻⁶ |
| (Wood units) | One single arteriole | |
| | (200 μm in diameter): | |
| | 4.75-143 × 10⁻⁶ | |
| | (calculated from estimated | |
| | number of arterioles) | |

TABLE 2
Technical specifications of the data acquisition system
that is used to interrogate the microfluidics device.

| Parameters | Values |
|---|---|
| Noise level | 28 (original) −> 0.24 w/10 Hz |
| (μV) | low-pass filter |
| Sensitivity | 7.6 |
| (mmHg/mV/V) | |
| Resolution | 0.02 (w/o processing), 0.00018 |
| (mmHg) | (w/10 Hz low-pass filter) |
| Long-term electrical | 0.01 in 3 hours |
| stability/zero | |
| drift (mmHg) | |
| Long-term mechanical | 0.3 in 3 hours; |
| stability/full- | 2 in 24 hours |
| span drift (mmHg) | |
| Sampling rate | 1613 |
| (samples/second) | |

### Pulmonary Hypertension

Pulmonary hypertension (PH or PHTN) is an increase of blood pressure in the pulmonary artery, pulmonary vein, or pulmonary capillaries, together known as the lung vasculature, leading to shortness of breath, dizziness, fainting, leg swelling and other symptoms. Pulmonary hypertension can be a severe disease with a markedly decreased exercise tolerance.

The signs/symptoms of pulmonary hypertension are consistent with the following: shortness of breath, chest pain, increased heartbeat, pain on the right side of the abdomen, poor appetite, lightheadedness, swelling (e.g., legs/ankles), and cyanosis. A detailed family history is established to determine whether the disease might be hereditary. A history of exposure to drugs such as cocaine, methamphetamine, ethanol (leading to cirrhosis), and tobacco is considered significant.

A physical examination is performed to look for typical signs of pulmonary hypertension, including a split S2, and loud P2 (pulmonic valve closure sound). Signs of systemic congestion resulting from right-sided heart failure are jugular venous distension, pedal edema, ascites, hepatojugular reflux, and clubbing. Evidence of tricuspid insufficiency and pulmonic regurgitation is also sought and, if present, is consistent with the presence of pulmonary hypertension.

The pathogenesis of pulmonary arterial hypertension involves the narrowing of blood vessels connected to and within the lungs. This makes it harder for the heart to pump blood through the lungs, much as it is harder to make water flow through a narrow pipe as opposed to a wide one. Over time, the affected blood vessels become stiffer and thicker, in a process known as fibrosis. This further increases the blood pressure within the lungs and impairs their blood flow. In common with other types of pulmonary hypertension, the increased workload of the heart causes hypertrophy of the right ventricle, making the heart less able to pump blood through the lungs, ultimately causing right heart failure. The right ventricle is normally part of a low-pressure system, with pressures that are lower than those that the left ventricle normally encounters. As such, the right ventricle cannot cope as well with higher pressures, and although hypertrophy of the heart muscle helps initially, it ultimately leads to a situation where the right ventricular muscle cannot get enough oxygen to meet its needs and right heart failure follows. As the blood flowing through the lungs decreases, the left side of the heart receives less blood. This blood may also carry less oxygen than normal. Therefore, it becomes harder and harder for the left side of the heart to pump to supply sufficient oxygen to the rest of the body, especially during physical activity.

Pathogenesis in pulmonary hypertension owing to left heart disease is completely different in that constriction or damage to the pulmonary blood vessels is not the issue. Instead, the left heart fails to pump blood efficiently, leading to pooling of blood in the lungs and backpressure within the pulmonary system. This causes pulmonary edema and pleural effusions.

In hypoxic pulmonary hypertension, the low levels of oxygen are thought to cause narrowing of the pulmonary arteries. This phenomenon is called hypoxic pulmonary vasoconstriction and it is initially a protective response designed to stop too much blood flowing to areas of the lung that are damaged and do not contain oxygen. When the damage is widespread and prolonged, this hypoxia-mediated vasoconstriction occurs across a large portion of the pulmonary vascular bed.

In chronic thromboembolic pulmonary hypertension, the blood vessels are blocked or narrowed with recurrent blood clots, and these clots can lead to release of substances that cause the blood vessels to constrict. This combination of blocked or narrowed vessels and vasoconstriction once again increases the resistance to blood flow and so the pressure within the system rises.

The molecular mechanism of pulmonary arterial hypertension (PAH) is not well understood, but it is believed that the endothelial dysfunction results in a decrease in the synthesis of endothelium-derived vasodilators such as nitric oxide and prostacyclin. Moreover, there is a stimulation of the synthesis of vasoconstrictors such as endothelin, thromboxane, and vascular endothelial growth factor (VEGF). These result in a severe vasoconstriction, and smooth muscle and adventitial hypertrophy—changes that are characteristic of patients with PAH.

In normal conditions, the nitric oxide synthase produces nitric oxide from L-arginine in presence of oxygen. Adenylate-cyclase and gualynate-cyclase are activated in presence of nitric oxide and these enzymes produce cAMP and cGMP respectively. The cGMP is produced by a type of guanylate cyclase (which is a kind of pyrophosphate-liase cyclase): the soluble guanylate cyclase (or sGC) that catalyzes the formation of cGMP from GTP. sGC is a heterodimer made up of one α subunit and one β subunit in each chain. It also contains a prosthetic heme group, required for NO binding. The union of NO and sGC produces a conformational enzyme change that stimulates cGMP production.

In the vascular endothelium, cGMP activates cGMP kinase or PKG (protein kinase G), which is an enzyme that belongs to a type of serine/threonine—specific protein kinase. PKG is a dimer composed of two similar polypeptide chains that share a common molecular structure. Each subunit contains a catalytic domain and regulatory domain. GMP-kinase activates potassium channels and subsequently the inhibition of calcium channels. Thus, this process leads to a reduction of intracellular calcium and finally a vasodilation.

Phosphodiesterase type V (PDE5), which is abundant in the pulmonary tissue, is a metalohydrolase that hydrolyzes the cyclic bond of cGMP in the presence of divalent cations (Zn²⁺). Actually, Zn²⁺ union is necessary for PDE5 activity. In the N-terminal region (regulatory domain) of PDE5 there is an amino acid sequence (residues 142-526) that joins cGMP. This sequence of PDE5 is divided in two domains; GAF-A and GAF-B; but only GAF-A has the necessary affinity to bind cGMP. This union increases the catalytic activity and it is stabilized by a close serine phosphorylation (performed by a kinase). Consequently, the concentration of cGMP decreases and the vasodilation is stopped. Patients with PAH produce less NO and others vasodilators and produce more vasoconstrictors. Consequently, this molecular pathway does not work properly and it results in a constant vasoconstriction. For this reason, NO and PDE5 inhibitors such as tadalafil or sildenafil are possible therapies.

Since the diagnosis of pulmonary hypertension dictates it can be of five major types, a series of tests must be performed to distinguish pulmonary arterial hypertension from venous, hypoxic, thromboembolic, or miscellaneous varieties. Further procedures are required to confirm the presence of pulmonary hypertension and exclude other possible diagnoses. These generally include pulmonary function tests; blood tests to exclude HIV, autoimmune diseases, and liver disease; electrocardiography (ECG); arterial blood gas measurements; X-rays of the chest (followed by high-resolution CT scanning if interstitial lung disease is suspected); and ventilation-perfusion or V/Q scanning to exclude chronic thromboembolic pulmonary hypertension. Clinical improvement is often measured by a “six-minute walk test”, i.e. the distance a patient can walk in six minutes. Stability and improvement in this measurement correlate with better survival.

Diagnosis of PAH requires the presence of pulmonary hypertension. Although pulmonary arterial pressure can be estimated on the basis of echocardiography, pressure measurements with a Swan-Ganz catheter through the right side of the heart provide the most definite assessment. Diagnosis of PAH requires right-sided cardiac catheterization; a Swan-Ganz catheter can also measure the cardiac output, which is far more important in measuring disease severity than the pulmonary arterial pressure. Normal pulmonary arterial pressure in a person living at sea level has a mean value of 8-20 mm Hg (1066-2666 Pa) at rest. Pulmonary hypertension is present when mean pulmonary artery pressure exceeds 20-25 mm Hg (3300 Pa) at rest. A physical examination is performed to look for typical signs of pulmonary hypertension. These include altered heart sounds, such as a second heart sound, a loud P2 or pulmonic valve closure sound (part of the second heart sound), and pulmonary regurgitation. Other signs include an elevated jugular venous pressure, peripheral edema (swelling of the ankles and feet), ascites (abdominal swelling due to the accumulation of fluid), hepatojugular reflux, and clubbing.

Treatment of pulmonary hypertension is determined by whether the PH is arterial, venous, hypoxic, thromboembolic, or miscellaneous. Treatment may include optimization of left ventricular function by the use of diuretics, digoxin, and blood thinners, or to repair/replace the mitral valve or aortic valve. Patients with left heart failure or hypoxemic lung diseases (groups II or III pulmonary hypertension) should not routinely be treated with vasoactive agents including prostanoids, phosphodiesterase inhibitors, or endothelin antagonists, as these are approved for the different condition called pulmonary arterial hypertension. To make the distinction, doctors at a minimum will conduct cardiac catheterization of the right heart, echocardiography, chest CT, a six-minute walk test, and pulmonary function testing. Using treatments for other kinds of pulmonary hypertension in patients with these conditions can harm the patient and wastes substantial medical resources. High dose calcium channel blockers are useful in only 5% of IPAH patients who are vasoreactive by Swan-Ganz catheter. Unfortunately, calcium channel blockers have been largely misused, being prescribed to many patients with non-vasoreactive PAH, leading to excess morbidity and mortality. The criteria for vasoreactivity have changed. Only those patients whose mean pulmonary artery pressure falls by more than 10 mm Hg to less than 40 mm Hg with an unchanged or increased cardiac output when challenged with adenosine, epoprostenol, or nitric oxide are considered vasoreactive. Of these, only half of the patients are responsive to calcium channel blockers in the long term.

Many pathways are involved in the abnormal proliferation and contraction of the smooth muscle cells of the pulmonary arteries in patients with pulmonary arterial hypertension. Three of these pathways are important since they have been targeted with drugs—endothelin receptor antagonists, phosphodiesterase type 5 (PDE-5) inhibitors, and prostacyclin derivatives.

Prostacyclin (prostaglandin 12) is commonly considered the most effective treatment for PAH. Epoprostenol (synthetic pros tacyclin) is given via continuous infusion that requires a semi-permanent central venous catheter. This delivery system can cause sepsis and thrombosis. Prostacyclin is unstable, and therefore has to be kept on ice during administration. Since it has a half-life of 3 to 5 minutes, the infusion has to be continuous, and interruption can be fatal. Other prostanoids have therefore been developed. PDE5 inhibitors are believed to increase pulmonary artery vasodilation, and inhibit vascular remodeling, thus lowering pulmonary arterial pressure and pulmonary vascular resistance.

Atrial septostomy is a surgical procedure that creates a communication between the right and left atria. It relieves pressure on the right side of the heart, but at the cost of lower oxygen levels in blood (hypoxia). Lung transplantation cures pulmonary arterial hypertension, but leaves the patient with the complications of transplantation, and a post-surgical median survival of just over five years. Pulmonary thromboendarterectomy (PTE) is a surgical procedure that is used for chronic thromboembolic pulmonary hypertension. It is the surgical removal of an organized thrombus (clot) along with the lining of the pulmonary artery; it is a very difficult, major procedure that is currently performed in a few select centers.

### Pharmaceutical Therapeutics

The invention provides pharmaceutical compositions for use as a therapeutic. In one aspect, the composition is administered systemically, for example, formulated in a pharmaceutically acceptable buffer such as physiological saline. Preferable routes of administration include, for example, instillation into the bladder, subcutaneous, intravenous, intraperitoneal, intramuscular, or intradermal injections that provide continuous, sustained levels of the composition in the patient. Treatment of human patients or other animals is carried out using a therapeutically effective amount of a therapeutic identified herein in a physiologically acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia or infection. Generally, amounts will be in the range of those used for other agents used in the treatment of other diseases associated with neoplasia or infection, although in certain instances lower amounts will be needed because of the increased specificity of the compound. A compound is administered at a dosage that enhances an immune response of a subject, or that reduces the proliferation, survival, or invasiveness of a neoplastic cell as determined by a method known to one skilled in the art.

### Formulation of Pharmaceutical Compositions

The administration of compositions for the treatment of a condition associated with endothelial dysfunction may be by any suitable means that results in a concentration of the therapeutic that, combined with other components, is effective in ameliorating, reducing, or stabilizing a condition associated with endothelial dysfunction. The composition may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intravesicularly or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice or nonhuman primates, as a skilled artisan recognizes it is routine in the art to modify the dosage for humans compared to animal models. In certain embodiments it is envisioned that the dosage may vary from between about 0.1 μg compound/kg body weight to about 5000 μg compound/kg body weight; or from about 1 μg/kg body weight to about 4000 μg/kg body weight or from about 10 μg/kg body weight to about 3000 μg/kg body weight. In other embodiments this dose may be about 0.1, 0.3, 0.5, 1, 3, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 μg/kg body weight. In other embodiments, it is envisaged that doses may be in the range of about 0.5 μg compound/kg body weight to about 20 μg compound/kg body weight. In other embodiments the doses may be about 0.5, 1, 3, 6, 10, or 20 mg/kg body weight. Of course, this dosage amount may be adjusted upward or downward, as is routinely done in such treatment protocols, depending on the results of the initial clinical trials and the needs of a particular patient.

Pharmaceutical compositions are formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Kits or Pharmaceutical Systems

Pharmaceutical compositions may be assembled into kits or pharmaceutical systems for use in treating a condition associated with endothelial dysfunction. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Modification and Validation of an Elastomeric Model of Pulmonary Vascular Smooth Muscle Cell Alignment and Function

One of the main concerns in vascular engineering is mimicking the three dimensional vascular architecture found in native tissues in the human body (Griffith L. G., and Naughton G., Science, 2002. 295:1009-14). Pulmonary microvessels are comprised primarily of longitudinally aligned intimal monolayer of endothelium (EC) surrounded by a medial layer of oblique and circumferentially aligned smooth muscle cells (SMC)(Isenberg, B. C., et al., Circ Res, 2006. 98:25-35; Lee, A. A., et al., J Biomech Eng, 2002. 124:37-43). In native blood vessels, SMC tend to adopt a spindle-like morphology and exhibit a contractile phenotype. However, SMC maintained in tissue culture condition adopt more of a round-shaped morphology and a synthetic phenotype (Shanahan C. M., and Weissberg P. L., Arterioscler Thromb Vasc Biol, 1998. 18:333-8), similar to those found in atherosclerosis (Chai, S., et al., Circ Res, 2005. 96:583-91) and intimal hyperplasia (Newby A. C., and Zaltsman A. B., J Pathol, 2000. 190:300-9). Thus, it is fundamental to consider the alignment, orientation and phenotypic presentation of smooth muscle cells in vessel bioengineering. Once the cellular components of the platform are in place, it is imperative that there is a cell-cell communication within the prototype. It has been demonstrated that EC and SMC form direct connections via cellular projections through the membrane pores in a vascular cell co-culture system (Isakson B. E., and Duling B. R., CircRes, 2005. 97:44-51; Isakson, B. E., et al., Circ Res, 2007. 100:246-54; Heberlein, K. R., et al., Circ Res, 2010. 106:1092-102). Furthermore, the role of these gap junctions in NO transmission from EC to SMC has been studied, and enrichment of hemoglobin has been shown to facilitate this process (Straub, A. C., et al., Nature, 2012. 491:473-7). The details of these relationships in human pulmonary microvessels are as yet unknown. Additionally, experiments were conducted using CORNING® TRANSWELL® membranes (Sigma Aldrich, St. Louis, Mo.) with a substantially thicker membrane than the IEL in blood vessels. The prototype described herein facilitates cell-cell communication through finely tuned micropores. Topographic modification on the surface of PDMS sheets facilitates SMC alignment along the direction of the wrinkles.

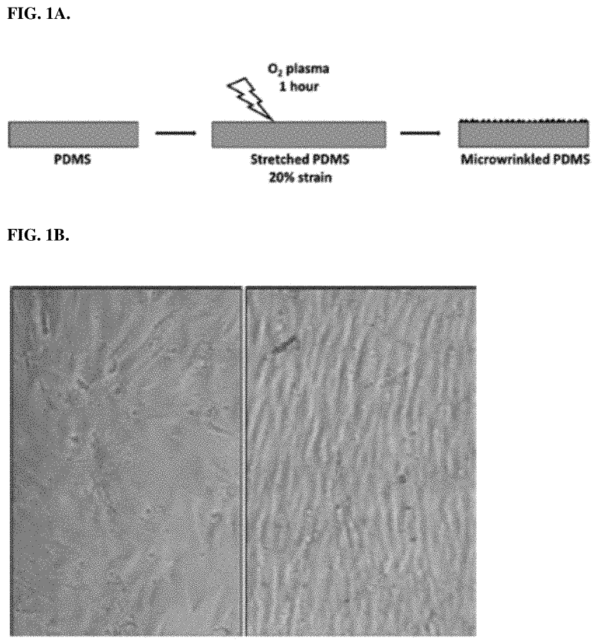

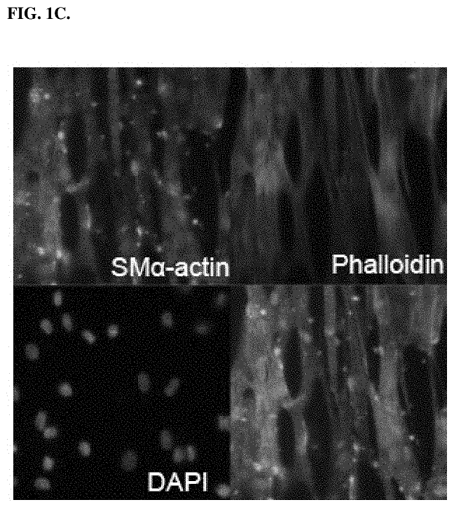

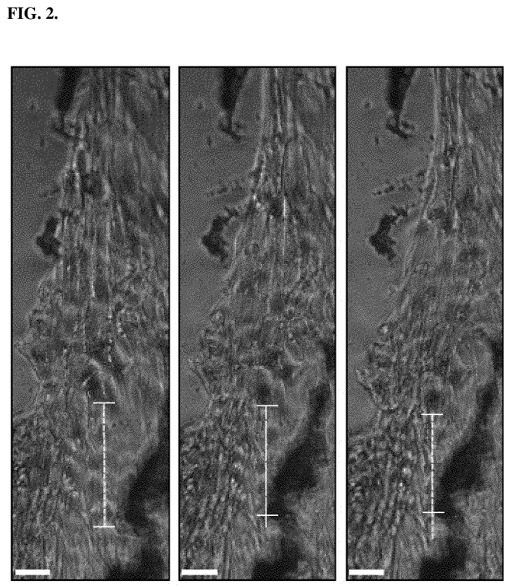

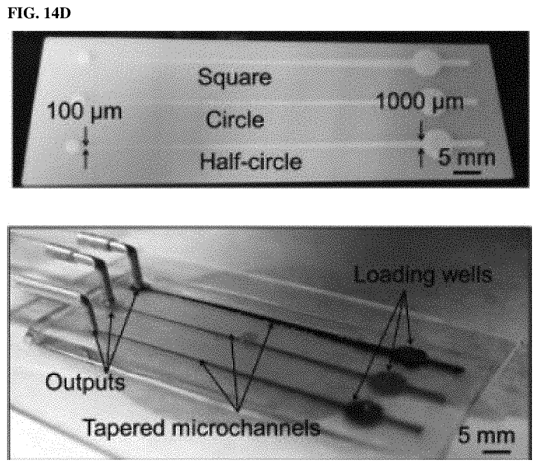

The data described herein show that PDMS membranes exposed to stretch followed by oxygen plasma etching produce consistent unidirectional wrinkles proportional to the direction of the strain. A 6 μm PDMS film was stretched by 20% with a uniaxial stretching system, followed by exposure to oxygen plasma (Plasma Etcher Inc.) at 80 W for 60 minutes to generate wrinkles (FIG. 1A-FIG. 1C and FIG. 16). HPASMC seeded on wrinkled PDMS showed alignment of smooth muscle cells parallel to the long axis of the wrinkles. Moreover, aligned HPASMC on wrinkles exhibited stretched morphology with characteristics of smooth muscle cells in a contractile phenotype found in blood vessels. To test the contractility of aligned HPASMC, HPASMC were exposed to angiotensin II (1 μM; Sigma Aldrich, St. Louis, Mo.) for 10 minutes. HPASMC demonstrated time-dependent contraction as shown by the fiduciary marks on the dotted vertical lines in FIG. 2. A more quantitative measurement of SMC contraction is employed as described in the Experimental Design section. To investigate whether SMC exhibit a contractile phenotype, SMC were cultured on coverslips coated with the ECM proteins, fibronectin, or type 1 collagen. SMC exhibited markedly higher expression of the contractile phenotype marker smoothelin on type 1 collagen-coated coverslips, along with an enlarged, elongated morphology, characteristics of contractile SMC (FIG. 3A-3B).

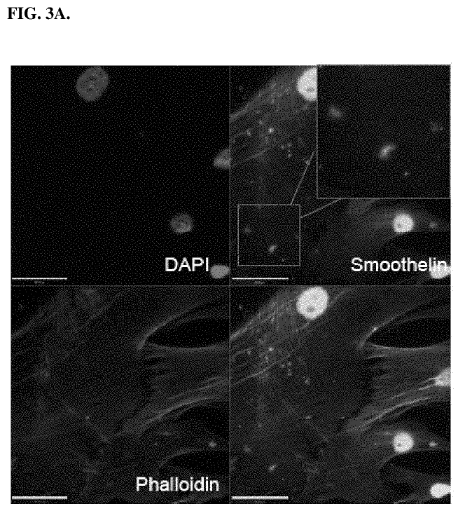

SMC relaxation is also measured on the microwrinkled elastomer. Blood vessels relax in response to blue light exposure in an endothelium independent manner (Sikka, G., et al., Proc Natl Acad Sci USA, 2014. 111:17977-82). As described herein, the relaxation observed with blue light exposure occurs via changes in actin-myosin interactions in the SMC. Indeed, almost a complete downregulation of ppMLC (diphosphorylation of the myosin light-chain) in HPASMC following exposure to blue light (FIG. 4) was observed. The advantages of using blue light-induced SMC relaxation are that it is endothelium-independent, non-pharmacological, rapid, and reversible. This phenomenon is utilized to measure smooth muscle relaxation on microwrinkled elastomer as a functional readout.

### Design of the Elastomer Surface to Attain Cellular Alignment Using Lithography

PDMS membranes of varying thickness (5-10 μm) are subjected to uni-axial stretch to 120% of their original dimension for 1 hour. The elastomers are placed into the plasma vacuum chamber to create a stiff, glass-like surface layer and strain-dependent wrinkle patterns on the surface (FIG. 1A). Varying stretch conditions (20%-50%) were used to change the depth and size of the wrinkles to achieve desired cell alignment. Furthermore, thickness of the PDMS membrane was controlled such that contraction produced by SMC shrinks the overall size of the membrane to quantify the extent of contraction. Depth and spacing of the wrinkles are quantified using scanning electron microscopy (Leong, T. G., et al., Small, 2010. 6:792-806). SMC contraction is quantified by measuring the change in spacing between imprinted grooves on the elastomer using nanoimprint lithography (Jaehyun, P., et al., Nano/Micro Engineered and Molecular Systems (NEMS), IEEE10th International Conference on, 2015. 233-237).

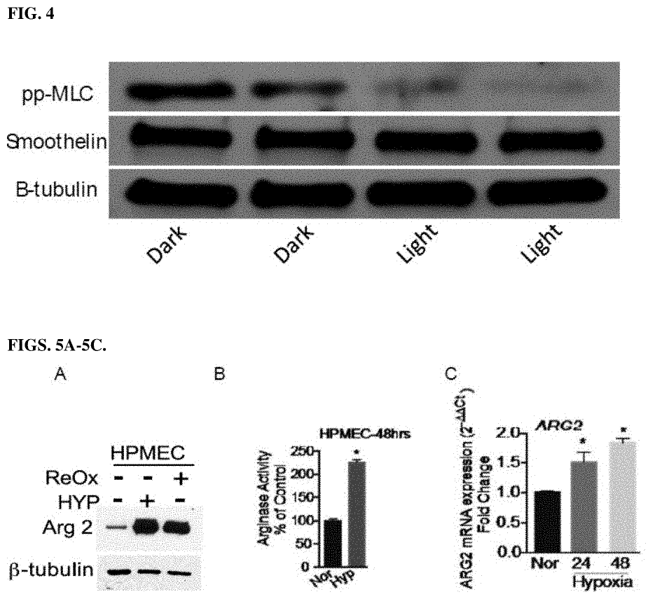

HPMEC were subjected to mechanical stretch that recapitulated a fetal breathing pattern with 15% equibiaxial stretch at 60 cycles/min (1 Hz) for 6 hours. Nitric oxide was measured at various time points (30 mins-6 hrs) and showed gradual increase in NO production with duration of stretch. HPMEC exposed to stretch with a pattern similar to that of fetal breathing demonstrate increased NO production in a time-dependent manner (see FIG. 6A). Stretch also induces eNOS phosphorylation with a concomitant decrease in arginase impression. HPMEC were exposed to equibiaxial stretch for 6 hr and eNOS phosphorylation and arginase 2 expression were detected by western blotting (see FIG. 6B).

### Smooth Muscle Cell Phenotype Alignment and Contraction

Human pulmonary artery smooth muscle cells (HPASMC) are seeded on sterilized wrinkled PDMS sheets. Type 1 collagen or laminin is used as a coating material. To investigate the contractibility of the HPASMCs cultured on the wrinkled PDMS membrane, expression of smooth muscle contractile phenotype markers such as SM-actin and smoothelin-1 is evaluated by immunofluorescence and western blotting. Alignment of HPASMCs is determined by measuring the angle between cell's major axis and the direction of microwrinkles ranging from 0 (a cell perfectly aligned with the direction of the microwrinkle) to 90° (a cell perpendicular to the direction of the microwrinkle) (Choi, J. S., et al., Biomaterials, 2014. 35:63-70). The contractility of HPASMCs cultured on wrinkled sheets is also measured. Confluent cells on wrinkled sheets are treated with contractile agents such as endothelin-1 (10 pM, Sigma) or angiotensin II (1 μM, Sigma) (Cain, A. E., et al., Hypertension, 2002. 39:543-9). Changes in the spacing of the grooves are measured using microscopy. For more precise measurement of SMC contraction, noncontact optical interferometric technique is used to quantify the fluctuation of PDMS membranes in response to the treatment of contractile agents (Park, Y., et al., ProcNatl Acad Sci USA, 2010. 107:6731-6). The idea is that contraction imposed by SMC alters the elasticity of the elastomer which is successfully measured by optical interferometry (Park, Y., et al., ProcNatl Acad Sci USA, 2010. 107:6731-6).

As described herein, the data clearly demonstrate that the stretching-releasing process creates a parallel wrinkled pattern in the PDMS that is perpendicular to the direction of stretch. Imaging of the HPASMCs confirms that wrinkles produce alignment of the cells parallel to the direction of wrinkles. In the event that SMC contraction is not sufficient to deform these microfabricated PDMS membranes due to their thickness (5-10 μm) and elastic modulus, boronic acid-functional poly(amido) amines may be used to produce thinner biocompatible elastomeric sheets (Hujaya, S. D., et al., Pharm Res, 2015. 32:3732-45). Pulmonary arterial smooth muscle cells are used as described herein, and these may be functionally distinct from pulmonary microvascular smooth muscle cells. SMC subpopulations in the pulmonary arterial circulation are reported to exhibit heterogeneity in phenotype, growth, and matrix producing capabilities (Frid, M. G., et al., Arterioscler Thromb Vasc Biol, 1997. 17:1203-9). This issue is addressed by isolating pulmonary microvascular smooth muscle cells from explanted human lungs.

The lithographic approach described herein (FIG. 1A) allows for the geometric confinement of smooth muscle cells in a physiologically relevant parallel alignment. The functional studies with SMC contraction indicate the functional relevance of cell alignment and geometry. Alternative microfabrication techniques, such as 3D printing, augment the geometrical complexity of the internal elastic lamina between the EC and SMC layers.

PAH is a serious challenge to public health that has yet to be adequately addressed with effective therapies. As described herein, the examples harness expertise in chemical engineering and vascular biology, and employ an in vitro model that incorporates the cellular constituents and microenvironmental features of human lung microvasculature to study nitric oxide signaling regulation in PAH. The interdisciplinary nature of the compositions differentiates this approach from others and leads to significant breakthroughs not just in fundamental knowledge but also tangible patient outcomes in the future.

### Example 3: Validation of Self-Folding 3D Tissue Culture System

Microfabrication techniques were employed to produce biodegradable, thin film scaffolds. Silicon oxides and germanium are first used as scaffold materials to test biocompatibility of these materials. Surface modification with matrix proteins (fibronectin or laminin) is tested to optimize cell growth. Cell longevity, density, and expression of cell specific biomarkers, including nitric oxide production, are used as parameters to assess compatibility of the materials. The quality of cell growth on 3D scaffolds with different radii of curvature and rigidity is studied. Endothelial cells and smooth muscle cells grown on these systems are assessed for expression of tissue-specific markers using immunohistochemistry Immunohistochemistry analysis of co-culture of endothelial and smooth muscle cells is performed. Laser scanning confocal microscopy is utilized to quantitatively assess protein marker expression and localization.

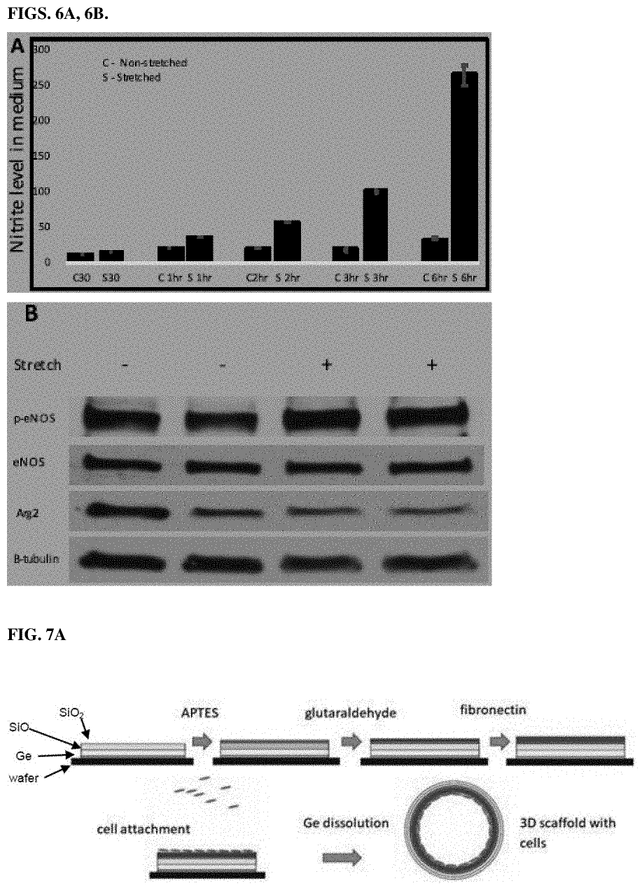

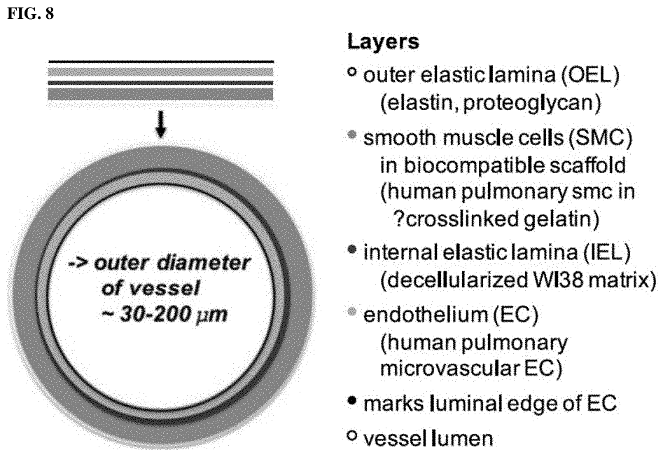

Microfabrication techniques were employed to make thin film scaffolds. This approach allowed for the possibility of obtaining reproducible results in a high-throughput manner. Thin films of germanium, silicon monoxide (SiO) and silicon dioxide (SiO₂) were sequentially deposited using physical vapor deposition. SiO/SiO2 Finite elemental analysis software, ABAQUS, was used to determine the appropriate thickness that promotes folding of the films for specific dimensions. Initial studies were done in 700 nm SiO and 700 nm SiO2 bilayer films of circular geometry with diameters that range from 1 mm to 3 mm Germanium was used as the sacrificial layer because of the presence of native GeO2, which dissolves in water. After film fabrication, film surfaces were modified with APTES and glutaraldehyde to covalently immobilize fibronectin. Then the films were seeded with human aortic endothelial cells that had been transformed with human telomerase reverse transcriptase (hTERT HAEC) (see FIGS. 7A-7C). The films are released upon dissolution of Ge and subsequently self-fold into tubular structures with the cells growing on the walls of the lumen. FIG. 9A shows tubes with different dimensions seeded with cells. The outer diameter of the tubes ranges from 60 to 380 μm, with film thickness of 1.4 μm. From these values the inner diameter of tubes are estimated to be from 57 to 377 μm. Hence, structures closely mimic the diameter of in vivo arterioles with lumen diameters that range from tens to hundreds of microns (Barreto-Ortiz, S. F., et al., FASEB J 2015, 29 (8), 3302-14; Mayrovitz, H. N. and Roy, J., Am J Physiol 1983, 245 (6), H1031-8; Kurbel, S., et al., Adv Physiol Educ 2009, 33 (2), 130-1). The viability of growing cells in 3D scaffolds was tested using Live/Dead assay after culturing for 8 days (see FIG. 9B and FIG. 9C) and demonstrated that cells are viable.

The cell density across the tube was further assessed to determine whether the cells were growing evenly in the tube. Cell density was analyzed using FIG. 9C. Cells were counted at 8 random areas across the tubes. As described herein, the results show even cell density across the tube and similar density between the two tubes (see Table 3) with a mean cell density between 21-24 cells/4000 m2.

TABLE 3
Cell density analysis on 3D scaffolds

| | Tube 1 | Tube 2 |
|---|---|---|
| (N = 8) Average Cells/40000 μm² | 23.9 | 20.9 |
| standard deviation | ±5.34 | ±2.62 |
| standard error of the mean | ±1.89 | ±0.93 |

Human pulmonary artery smooth muscle cells are cultured in the 3D scaffolds. Gene expression of smooth muscle cells has been shown to be affected by the type of extracellular matrix used in vitro (Hirst, S. J., et al., Am J Respir Cell Mol Biol 2000, 23 (3), 335-44). ECM matrix is easily modified in 3D scaffolds to promote adhesion and grow different cell types.

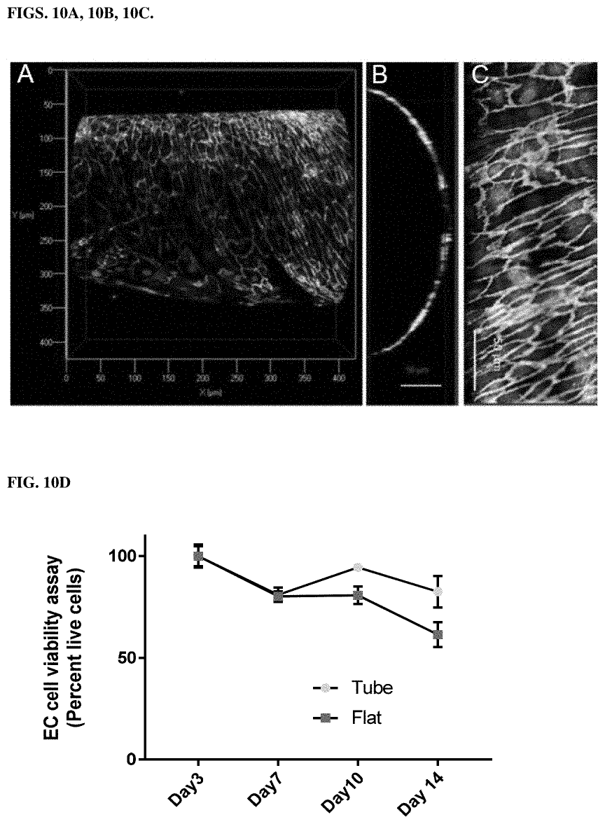

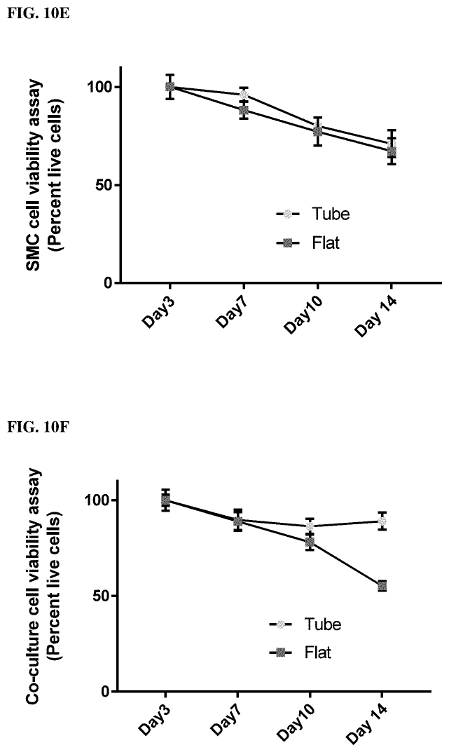

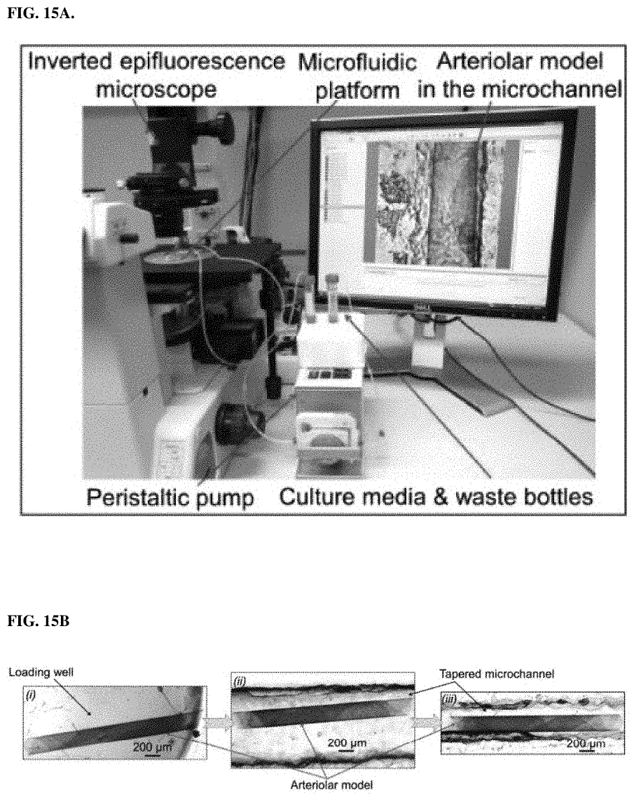

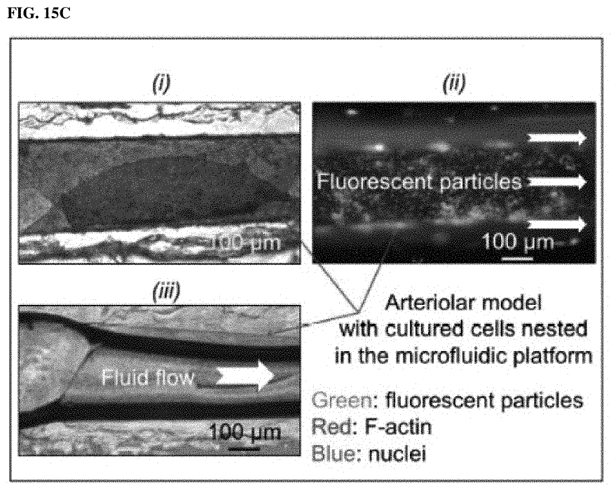

hTERT HAEC growing for 28 days were immunostained to visualize the presence of VE-cadherin, F-actin and nucleus (see FIGS. 11A-11B, 12A-12C, and 17A-17C). Tight monolayers were formed, as shown by the presence of VE-cadherin (see FIG. 17A-17C)—indicating that these scaffolds support cell growth. The orthogonal view shows the lumen of the tube lined with endothelial cells that mimics the geometry and spatial organization of arterioles. Smooth muscle cells and endothelial cells are also co-cultured in these scaffolds.

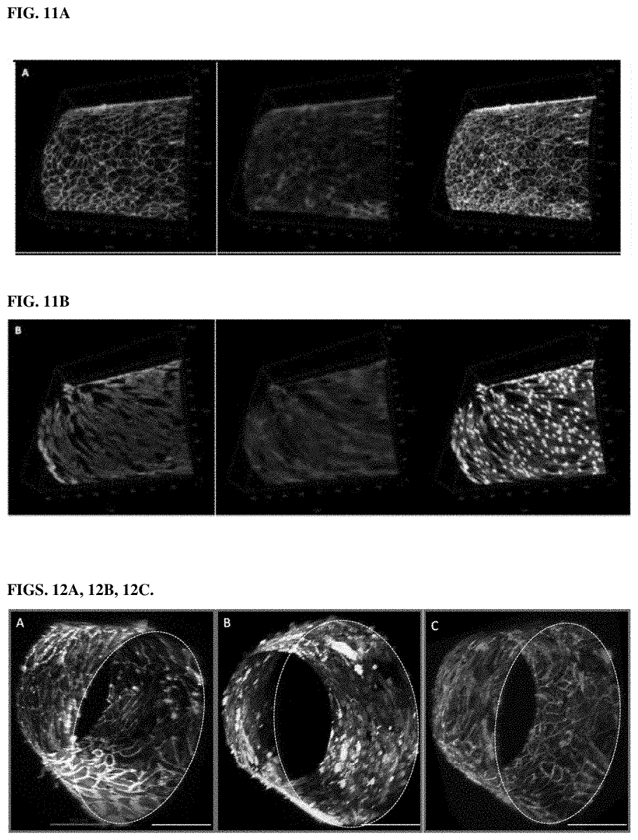

Endothelial cells have many functions including the production of nitric oxide (NO). NO is a vasoactive agent important for modulating the vascular smooth muscle tone (Davidge, S. T., et al., Circ Res 1995, 77 (2), 274-83). The confluent cells growing for 2 months in the tubes were tested for NO production to determine endothelial function and viability. To qualitatively assess the NO production, 50 μL of the conditioned-media was analyzed using Seivers NO analyzer. FIGS. 13A-13C show NO production by hTERT HAEC with and without calcium ionophore stimulation. A 7- to 35-fold increase of NO was observed when HAEC growing in 3D scaffolds was stimulated with 1 μM A23187 calcium ionophore for 30 minutes. Others have reported twofold NO increase after 60-minute incubation of HAEC in 1 μM ionomycin grown on well plates (Cosentino, F., et al., Circulation 1997, 96 (1), 25-8). Using bovine coronary microvascular endothelial cells, a 3-fold increase was observed when stimulated with 1 μM A23187 calcium ionophore for 5 hours (Davidge, S. T., et al., Circ Res 1995, 77 (2), 274-83). Stimulation of human coronary artery endothelial cells with 20 ng/mL of hepatocyte growth factor, reported to induce NO production, (Uruno, A., et al., HypertensRes2004, 27 (11), 887-95) resulted in twofold NO increase (Ueba, H., et al., Atherosclerosis 2005, 183 (1), 35-9). There is difficulty in direct comparison of results with reported observations due to differences in methodology, analysis, and cell types. Nevertheless, it is evident that the results described herein showed higher increase in NO production upon stimulation compared to previous reports. The higher increase in NO production warrants further investigation if 3D geometry affects HAEC NO production. These data suggest that the hTERT HAECs in 3D scaffolds remained functional and viable after 2 months of culture. This observation is favorable for longitudinal study of pathobiology and therapy of pulmonary hypertension

In summary, the results described herein have shown the feasibility of growing cells in self-folding scaffolds modified with fibronectin. The cells grew to confluency in 3D scaffolds and exhibited endothelial markers, VE-cadherin and F-actin. It is noteworthy that the endothelial cells remained viable and functional after 2 months even in static culture as demonstrated by NO production. Interestingly, hTERT HAECs responded with higher increase in NO production after calcium ionophore stimulation compared to other observations. Understanding of the surface chemistry of silicon allows the customization of the ECM needed to promote adhesion and proliferation of different cell types. Bypassing the need to use chemical etchants to remove the sacrificial layer and initiate folding maintains high cell viability. Furthermore, application of microfabrication techniques enabled multiple films with different dimensions in a high-throughput manner Overall, these results described herein demonstrate the biomimetic tissue-engineered human arterioles for in vitro platform of studying pulmonary hypertension.

### Rationale

Biodegradable, bioactive 3D scaffolds made from biocompatible materials are some of the important considerations for biomimetic tissue-engineering (Jung, Y.-G., et al., Journal of Materials Research 2004, 19 (10), 3076-3080). Microfabricated thin film SiO/SiO2 bilayer modified with appropriate extracellular matrix promotes cell adhesion and sustains normal growth and function.

### Design

### Characterization of Scaffold Mechanical Property.

Thin SiO/SiO2 film scaffolds degrade in aqueous environment. Dissolution of thin SiO/SiO2 films in aqueous environments was observed previously to be 1-2 nm/day and 10 nm/day (Malachowski, K., et al., Nano Lett 2014, 14 (7), 4164-70; Arayanarakool, R., et al., Lab Chip 2015, 15 (14), 2981-9). The difference in results may be attributed to the difference in physical characteristics and aqueous environment used by the different groups. Physical characteristics, such as porosity and hardness, of the films depend on the parameters used in fabrication such as temperature and deposition rate (Jung, Y.-G., et al., Journal of Materials Research 2004, 19 (10), 3076-3080). The Young's modulus of SiO and SiO2 reported in literature were 77 and 75 GPa, respectively (Malachowski, K., Nano Lett 2014, 14 (7), 4164-70). While the reported Young's modulus of the radial artery wall material at mean arterial pressure in normotensive patients to be around 2.68 MPa (Laurent, S.; et al., Arterioscler Thromb 1994, 14 (7), 1223-31). Even though the Young's modulus of the film is greater than the reported Young's modulus for the arterial wall, it is important to remember that the film disintegrates. Hence, the mechanical properties of the films change overtime. The changes in the Young's modulus of the film upon prolonged exposure to aqueous solution and films with different dimensions are investigated using atomic force microscope. Softer materials such as biodegradable hydrogels and synthetic polymers are also used as scaffolds for cells if normal cell behavior is not observed in SiO/SiO2 scaffolds (Barreto-Ortiz, S. F., et al., FASEB J2015, 29 (8), 3302-14; BaoLin, G. and Ma, P. X., Sci China Chem 2014, 57 (4), 490-500). As shown in FIGS. 9A-9C, rolled tubes have “swiss roll” folding as shown by overlapped edges. The “swiss roll” folding may have an effect on tissue growth and at the same time designing new film dimensions allows the possibility of obtaining tubes without overlap.

### Protein Marker Expression Study of Single Cell Culture and Co-Culture in 3D Scaffolds.

Different cell cultures are examined in 3D such as human pulmonary artery smooth muscle cells (HPASMC) only, human pulmonary artery endothelial cells (HPAEC) only, and co-culture of HPASMC and HPAEC. These experiments are done in parallel and results are compared with experiments done in 2D cell culture. The results described herein support the hypothesis that SiO/SiO2 bilayer modified with appropriate extracellular matrix promotes cell adhesion and sustains normal growth. Even cell distribution on the scaffolds and expression of endothelial cell markers has been demonstrated as described herein. Several ECM such as laminin and collagen I are tested to determine if ECM will affect cell growth and protein expressions growing in 3D scaffolds (Senoo, H. and Hata, R., Kaibogaku Zasshi 1994, 69 (6), 719-33). Using immunohistochemistry and confocal microscopy, the presence and co-localization of specific cell markers such as VE-cadherin, —smooth muscle actin and smoothelin are evaluated. Endothelial cell expression of VE-cadherin indicates formation of gap junctions necessary for cell-cell communications. Smoothelin and -smooth muscle actin are contractile phenotype markers for smooth muscle cells (Beamish, J. A., et al., Tissue Eng Part B Rev 2010, 16 (5),467-9).

### Functional Study of Tissue-Engineered Arterioles.

Normal tissue function is as important as normal growth. Contraction output of smooth muscle cells has been reported to be dependent on cell shape (Alford, P. W., et al., Integr Biol (Camb) 2011, 3 (11), 1063-70). The effect of the 3D geometry on smooth muscle actin and nuclear orientation is evaluated using immunostaining and confocal microscopy (Serbo, J. V., et al., Adv Healthc Mater 2015). The correlation between cell morphology, protein expression, and contraction output is determined. Contraction output is analyzed using myograph or live microscopy. Cell morphology, protein expression, and contraction is compared between HPASMC only and co-culture of HPASM-HPAEC. Studies have shown that in co-culture, endothelial cells regulate smooth muscle cell contraction (Ge, D., et al., Acta Pharmacol Sin 2012, 33 (1), 57-65). Vasoactive factors that affect regulation of microvascular tone such as NO, cGMP and Krüppel-like factor 4 (KLF4) are quantitatively assessed (Yu, M., et al., Am J Physiol Heart Circ Physiol 2002, 282 (5), H1724-31; Shatat, M. A., et al., Am JRespir Cell Mol Biol 2014, 50 (3), 647-53). Western blot is used to quantify cGMP and KLF4. Increases in cGMP should correlate with increases in NO, if normal communication between endothelial and smooth muscle cells is present. 4,5-diaminofluorescein diacetate (DAF-2 DA) is used to detect and quantify NO production in situ. DAF-2 DA is hydrolyzed by cytosolic esterase, where the released DAF-2 is converted into fluorescent triazole derivative in the presence of NO (Kojima, H., et al., Anal Chem 1998, 70 (13), 2446-53). The fluorescence signal is normalized to the scanned surface area, thus eliminating the need for tedious quantification of endothelial cells for normalization (Gamez-Mendez, A. M., et al., PLoSOne 2015, 10 (9), e0138609). The results described herein show that hTERT HAECs are viable and functional for 2 months. This allows longitudinal examination of the effects of changes in chemical and mechanical stimuli on tissue growth and function.

### Example 4: Interrogate Effects of Physiologic Stress and Drugs on Tissue Function

These tubes are integrated into a microfluidics system that facilitates control of pressure and measurements of flow. Pharmacologic modulators of vasomotor tone are introduced, and the time course and dose responses of the biomimetic microvascular tissue are assayed. This work addresses the urgent need to develop in vitro systems that hasten the understanding of the pathobiology of pulmonary hypertension and perform drug discovery studies in physiologically relevant systems.

### Rationale

Shear flow and cyclic stretch are mechanical forces that affect mechanotransduction and subsequent cell behavior of endothelial and smooth muscle cells (Zheng, W., et al., Am J Physiol Heart Circ Physiol 2008, 295 (2), H794-800; Ochoa, C. D., et al., Am JRespir Cell Mol Biol 2008, 39 (1), 105-12). By introducing shear flow and cyclic stretch to tissue-engineered arterioles, physiologically relevant data in studying pathobiology and therapeutics of pulmonary hypertension is obtained.

### Design

### Effect of Shear Flow on Biomimetic Tissue Function and Behavior.

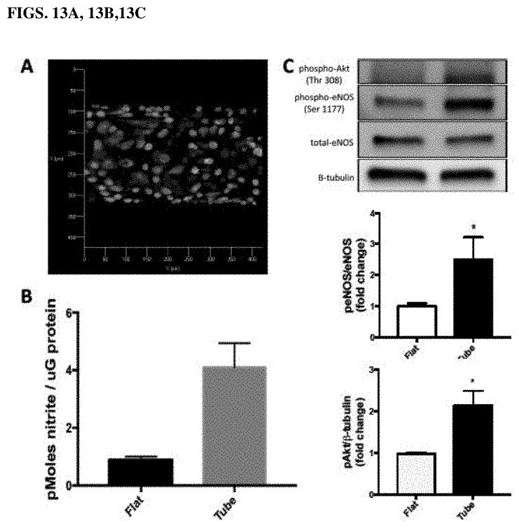

Shear flow is introduced into tissue-engineered arterioles using an integrated microfluidic system, as illustrated in FIG. 14A. Microfluidic components are composed of poly(dimethyl siloxane) (PDMS) using microfabrication or poly(methyl methcarylate) as described herein (Pagaduan, J. V., et al., Electrophoresis 2015, 36 (5), 813-7; Kim, L., et al., Lab Chip 2007, 7 (6), 681-94). The set-up includes a peristaltic pump that induces pulsatile flow. The tissue-engineered arterioles are cannulated into microfluidic culture chamber. For comparison, similar experiments are performed in parallel in static, standard 2D cell culture. Differences in cell morphology and protein expression are expected between tissues in static 2D culture and the integrated microfluidic system. Arterioles integrated into the microfluidic system exhibit in vivo like behavior. Protein expression of cell specific markers is also assessed. An anticipated challenge to overcome in the assembly of this microfluidics system is cannulating the small diameter tissue-engineered structures. One method is to custom-make cannulation pipettes from capillary tubes that match the diameter of the biomimetic arterioles (Socha, M. J. and Segal, S. S., JVisExp 2013, (81), e50759). Microsurgical glue such as fibrin glue is also an alternative to connect the biomimetic arterioles to the microfluidic system (Sacak, B., et al., JPlast Surg Hand Surg 2015, 49 (2), 72-6).

### Biomimetic Tissue Response on Vasoactive Drugs.

The microfluidic mixer module allows automated introduction of different drugs and concentrations to multiple arterioles located in the culture chamber. Biomimetic tissue response to epinephrine and acetylcholine is first used to test contraction and relaxation responses respectively (Kodama, Y., et al., JSmooth Muscle Res2010, 46 (4), 185-200; Bolton, T. B., Physiol Rev 1979, 59 (3), 606-718). The small footprint of the microfluidic mixer and culture chamber enables the use of existing live cell imaging microscopes. Live cell imaging and digital caliper is used to measure vasoconstriction or dilation of the vessels during the experiment. The modular design of the system permits addition of sensors to measure changes in flow velocity and pressure inside the microvessels (see FIG. 14B) in response to treatments. Automated recording of measurements over a period of time may be achieved by using commercially available systems that integrate data acquisition and digital display. The system, as described herein, is efficient and easy to use once optimized. Commercially available pulmonary hypertension drugs are also tested in this system for their effects on vessel caliber, nitric oxide production, flow and pressure. For example, 100 M of the commercial drug Bosentan (Tracleer, Actelion) (a dual endothelin receptor agonist) altered proliferation and smooth muscle alpha actin expression in fibroblasts (Bogatkeich, G. S., et al., Rheumatology 2012, S1:005). Sildenafil, a phosphodiesterase 5 inhibitor, (Schwebe, M., et al., Biochem Pharmacol 2015, 94 (2), 109-29) from Viagra (Pfizer) has been implicated in improved insulin signaling and may improve endothelial nitric oxide synthase activity (Mammi, C., et al., PLoSOne2011, 6 (1), e14542).

As described herein, this approach to biomimetic tissue engineering offers reproducible scaffolds with precisely controlled dimensions. Different adherent cells are easily used in this system because of the possibility of tailored surface modifications. These results indicate that the approach maintains cell viability, growth, and function for at least 2 months. Based on these results, the approach is applicable to longitudinal studies that are further advantageous for investigating pathobiology and therapies of pulmonary hypertension. The integrated microfluidic system described herein enables parallel study of tissue responses to mechanical forces and vasoactive drugs. The compositions described herein are vital to hastening the understanding of the pathobiology of pulmonary hypertension and enable drug discovery studies in physiologically relevant systems.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A composition comprising a biomimetic in vitro model of an arteriolar vessel comprising:
at least one of 1) human smooth muscle cells and 2) human endothelial cells;
wherein the vessel recapitulates one or more of the overall tubular geometry, morphometrics, extracellular matrix constituents, cellular morphology, cellular alignment, and functional heterotypic connections between the human smooth muscle cells and/or the human endothelial cells as compared to an in vivo arteriolar vessel.

2. A composition comprising a biomimetic in vitro model of an arteriolar vessel comprising:
at least one of 1) human smooth muscle cells and 2) human endothelial cells;
wherein the vessel recapitulates one or more of the extracellular matrix constituents, cellular morphology, cellular alignment, and functional heterotypic connections between the human smooth muscle cells and/or the human endothelial cells as compared to an in vivo arteriole vessel.

3. The composition of claim 1 wherein the composition comprises a biomimetic in vitro model of a small blood vessel, ocular vessel, renal vessel or coronary vessel.

4. The composition of claim 1 wherein the composition comprises a biomimetic in vitro model of a pulmonary vessel.

5. The composition of claim 1 wherein the composition comprises a) a biomimetic in vitro model of a pulmonary vessel and b) at least one of human pulmonary artery smooth muscle cells (HPASMC) and human pulmonary endothelial cells (HPMEC).

6. The composition of claim 1 wherein the composition comprises both human smooth muscle cells and human endothelial cells.

7. The composition of claim 1 wherein the in vitro model comprises a monolayer of HPMEC.

8. The composition of claim 1 further comprising elastomeric sheets, wherein the HPASMC and the HPMEC are upon the elastomeric sheets.

9. The composition of claim 8 wherein the elastomeric sheets are micro-wrinkled.

10. The composition of claim 8 wherein the elastomeric sheets comprise polydimethylsiloxane (PDMS) and/or boronic acid-functional poly(amido) amines.

11-21. (canceled)

22. A method of screening candidate compounds for treating pulmonary arterial hypertension (PAH) comprising:
inducing a composition (biomimetic composition or vessel) of claim 1 to exhibit PAH;
determining that the vessel has PAH;
administering a candidate compound to the biomimetic composition, and
determining whether the vessel has a reduction in PAH relative to a control vessel that does not have PAH.

23-27. (canceled)

28. A method of producing a biomimetic in vitro model of a pulmonary vessel comprising:
cultivating HPASMC on elastomeric sheets; and
adding monolayers of endothelial cells,
wherein the vessel recapitulates cellular morphology, cellular alignment, and functional heterotypic connections between the HPASMC and the endothelial cells as compared to an in vivo pulmonary vessel.

29. The method of claim 28 wherein the endothelial cells comprise HPMEC and microvasculature endothelial cells of the eye, kidney, and/or coronary circulation.

30. A composition comprising a biomimetic, three-dimensional (3D), in vitro tubular model of a pulmonary vessel comprising:
a biodegradable, self-folding scaffold comprising one or more cardiovascular cells or microvasculature cells from the eye, kidney or coronary circulation, wherein the cells are seeded on a substrate,
wherein the tubular model closely mimics the diameter of an in vivo pulmonary vessel, the tubular model comprises micropatterned fibronectin and/or laminin 3 and smooth muscle cells that are aligned on a patterned substrate, an internal elastic lamina, and a monolayer of endothelial cells, and the tubular model promotes cell-cell communication.

31-52. (canceled)

53. A method of screening a candidate compound for treating pulmonary arterial hypertension (PAH) or other microvasculature condition comprising:
inducing a composition (biomimetic composition or vessel) of claim 30 to exhibit PAH or abnormal vasomotor behavior,
determining that the vessel has PAH or abnormal vasomotor behavior,
administering a candidate compound to the biomimetic vessel, and
determining whether the vessel has a reduction in PAH or abnormal vasomotor behavior relative to a control vessel that does not have PAH or abnormal vasomotor behavior.

54. A device for modeling pulmonary circulation comprising:
a rectangular platform comprising an input port at an opposing end of an output port for circulation of flow;
a microchannel connecting the input and output port wherein the microchannel is tapered having a smaller diameter at the output port;
a loading well interposed between the input port and output port,
an upstream pressure readout port connected to the microchannel and situated after the loading well;
a downstream pressure readout port connected to the microchannel and situated prior to the output port.

55. The device of claim 54, wherein the input and output ports provide for circulation of flow.

56. The device of claim 54, wherein the loading well is provided for insertion of 3-dimensional (3D) model vessels.

57. The device of claim 54, wherein the pressure readout ports at the upstream and downstream ends of the microchannel are used to track systolic and diastolic pressures in real-time.

58. The device of claim 57, wherein pressure differences between the upstream and downstream pressure readout ports are a measure of vascular resistance across the microchannel.

59-61. (canceled)
